(19) United States
(12) Patent Application Publication
(10) Pub. No.: US 2013/0011538 A1
(43) Pub. Date: Jan. 10, 2013

(54) CRYSTAL OF MULTIVALENT METAL SALT OF MONATIN

(75) Inventor: Kenichi MORI, Kawasaki-shi (JP)

(73) Assignee: Ajinomoto Co., Inc., Chuo-ku (JP)

(21) Appl. No.: 13/492,140

(22) Filed: Jun. 8, 2012

Related U.S. Application Data
(60) Provisional application No. 61/494,639, filed on Jun. 8, 2011.

(51) Int. Cl. C07D 209/20; A23L 1/236

(52) U.S. Cl. 426/548; 548/495

(57) ABSTRACT

To provide a novel monatin crystal capable of forming a sweetener composition which is less likely to be degraded even when being exposed to high temperature and high humidity conditions in the coexistence of a reducing sugar. It was found that the object can be achieved by a crystal of a multivalent metal salt of (2R,4R)-monatin.

### CROSS REFERENCES TO RELATED APPLICATIONS

This application claims priority to U.S. Provisional Patent Application No. 61/494,639, filed on Jun. 8, 2011, which is incorporated herein by reference in its entirety.

### TECHNICAL FIELD

The present invention relates to a novel crystal of a multivalent metal salt of (2R,4R)-monatin. The present invention also relates to a sweetener composition containing the crystal. Further, the present invention relates to a sweetener composition containing a reducing sugar.

### Background Art

Monatin is a natural amino acid derivative isolated from the root bark of a plant (Schlerochiton ilicifolius) naturally grown in a region of the northern Transvaal of South Africa, and it has been reported by R. Vleggaar et al. that Monatin has a structure of (2S,4S)-2-amino-4-carboxy-4-hydroxy-5-(3-indolyl)-pentanoic acid ((2S,4S)-4-hydroxy-4-(3-indolylmethyl)-glutamic acid) (Non-patent document 1). Further, according to this document etc., the sweetness intensity of this (2S,4S) substance (natural-type monatin) derived from a natural plant has been reported to be 800 to 1400 times higher than that of sucrose. As a synthesis method of monatin, there have been reported several methods, however, many of them are related to a method for synthesizing a stereoisomeric mixture, and there have been almost no reports that 4 types of stereoisomers having the same chemical structural formula as natural-type monatin are synthesized and isolated as pure products, respectively, and their properties are examined in detail (Patent documents 1 to 3, Non-patent documents 2 to 3).

Recently, several studies have been made for a method for producing monatin (Patent documents 4 to 5), and further, as monatin crystals, several findings have been reported, however, there has been no description of Examples of crystals of a multivalent metal salt and an effect thereof (Patent documents 6 to 10).
[Patent document 1] ZA 87/4288
[Patent document 2] ZA 88/4220
[Patent document 3] U.S. Pat. No. 5,994,559
[Patent document 4] WO 2003-056026
[Patent document 5] WO 2003-059865
[Patent document 6] WO 2003-045914
[Patent document 7] US 2005-272939
[Patent document 8] JP-A-2005-154291
[Patent document 9] JP-A-2006-052213
[Patent document 10] JP-A-2010-155817
[Non-patent document 1] R. Vleggaar et al., J. Chem. Soc. Perkin Trans., 3095-3098, (1992)
[Non-patent document 2] Holzapfel et al., Synthetic Communications, 24(22), 3197-3211 (1994)
[Non-patent document 3] K. Nakamura et al., Organic Letters, 2, 2967-2970 (2000)

### DISCLOSURE OF THE INVENTION

### Problems that the Invention is to Solve

An object of the present invention is to provide a novel monatin crystal capable of forming a sweetener composition which is less likely to be degraded even when being exposed to high temperature and high humidity conditions in the coexistence of a reducing sugar.

### Means for Solving the Problems

The present inventors made intensive studies, and as a result, they found that the above-described object can be achieved by a crystal of a multivalent metal salt of (2R,4R)-monatin.

That is, the present invention includes the following aspects.

[1] A crystal of a multivalent metal salt of (2R,4R)-monatin.

[2] The crystal of a multivalent metal salt of (2R,4R)-monatin according to [1], wherein the multivalent metal salt is a divalent metal salt.

[3] The crystal of a multivalent metal salt of (2R,4R)-monatin according to [2], wherein the multivalent metal salt is an alkaline earth metal salt.

[4] The crystal of a multivalent metal salt of (2R,4R)-monatin according to [3], wherein the multivalent metal salt is at least one salt selected from calcium salts and magnesium salts.

[5] The crystal of a multivalent metal salt of (2R,4R)-monatin according to [4], wherein the crystal is a crystal of ((2R,4R)-monatin)₂ magnesium salt having characteristic X-ray diffraction peaks at diffraction angles (2θ±0.2°, CuKα) of 4.9°, 16.8°, 18.0°, and 24.6°.

[6] The crystal of a multivalent metal salt of (2R,4R)-monatin according to [4], wherein the crystal is a crystal of ((2R,4R)-monatin)₂ magnesium salt having characteristic X-ray diffraction peaks at diffraction angles (2θ±0.2°, CuKα) of any one of the following (1) to (3):

(1) 8.7°, 10.5°, 15.9°, 17.4°, 21.0°, and 25.6°,

(2) 8.9°, 11.2°, 15.0°, 17.8°, and 22.5°; and

(3) 4.9°, 16.8°, 18.0°, and 24.6°.

[7] The crystal of a multivalent metal salt of (2R,4R)-monatin according to [4], wherein the crystal is a crystal of ((2R,4R)-monatin)₂ magnesium salt having characteristic X-ray diffraction peaks at diffraction angles (20±0.2°, CuKα) of any one of the following (1) to (4):

(1) 7.5°, 10.3°, 11.2°, 16.0°, 18.1°, and 23.0°;

(2) 8.7°, 10.5°, 15.9°, 17.4°, 21.0°, and 25.6°;

(3) 8.9°, 11.2°, 15.0°, 17.8°, and 22.5°; and

(4) 4.9°, 16.8°, 18.0°, and 24.6°.

[8] The crystal of a multivalent metal salt of (2R,4R)-monatin according to [4], wherein the crystal is a crystal of ((2R,4R)-monatin)₂ calcium salt having characteristic X-ray diffraction peaks at diffraction angles (20±0.2°, CuKα) of either one of the following (1) and (2):

(1) 5.0°, 12.8°, 15.3°, 18.1°, and 23.7°; and

(2) 6.0°, 9.8°, 16.0°, 21.5° and 22.3°.

[9] The crystal of a multivalent metal salt of (2R,4R)-monatin according to [4], wherein the crystal is a crystal of ((2R,4R)-monatin)₂ calcium salt having characteristic X-ray diffraction peaks at diffraction angles (20±0.2°, CuKα) of any one of the following (1) to (3):

(1) 5.1°, 15.9°, 19.7°, and 22.3°;

(2) 5.0°, 12.8°, 15.3°, 18.1°, and 23.7°; and

(3) 6.0°, 9.8°, 16.0°, 21.5° and 22.3°.

[10] The crystal of a multivalent metal salt of (2R,4R)-monatin according to any one of [1] to [9], wherein the crystal has an enantiomeric excess of from 10 to 100% ee.

[11] The crystal of a multivalent metal salt of (2R,4R)-monatin according to any one of [1] to [10], wherein the crystal has a diastereomeric excess of from 10 to 100% de.

[12] The crystal of a multivalent metal salt of (2R,4R)-monatin according to any one of [1] to [11], wherein the crystal has a chemical purity of from 50 to 100% by mass.

[13] The crystal of a multivalent metal salt of (2R,4R)-monatin according to any one of [1] to [12], wherein the crystal has a sweetness intensity 200 times or more higher than an aqueous solution of 5% sucrose.

[14] A sweetener composition, comprising the crystal of a multivalent metal salt of (2R,4R)-monatin according to any one of [1] to [13].

[15] The sweetener composition according to [14], further comprising a reducing sugar.

[16] The sweetener composition according to [15], wherein the reducing sugar is dihydroxyacetone, glyceraldehyde, erythrulose, erythrose, threose, ribulose, xylulose, ribose, arabinose, xylose, lyxose, deoxyribose, psicose, fructose, sorbose, tagatose, allose, altrose, glucose, mannose, gulose, idose, galactose, talose, fucose, fucrose, rhamnose, sedoheptulose, lactose, maltose, turanose, cellobiose, maltotriose, or acarbose.

[17] The sweetener composition according to any one of [14] to [16], wherein the composition is in the form of a powder.

[18] The sweetener composition according to [14], further comprising a reducing sugar-producing substance.

[19] An oral product, comprising the crystal of a multivalent metal salt of (2R,4R)-monatin according to any one of [1] to [13].

[20] An oral product, comprising the sweetener composition according to any one of [14] to [18].

### Advantage of the Invention

It was found that by using a crystal of a multivalent metal salt of (2R,4R)-monatin, a sweetener composition which is less likely to be degraded even when being exposed to high temperature and high humidity conditions in the coexistence of a reducing sugar can be formed.

It became also possible to elucidate the utility and physical properties of stereoisomers thereof as sweeteners. Further, it became possible to provide oral products such as drinks, foods, pharmaceuticals, quasi drugs, and feeds, each containing a versatile stable and safe crystal of a multivalent metal salt of monatin. It is a matter of course that the present invention can also be applied to (2S,4S)-monatin.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is a powder X-ray diffraction chart of crystals of ((2R,4R)-monatin)₂ calcium salt pentahydrate after humidity conditioning (Example 1).

FIG. 2 is an optical microphotograph of crystals of ((2R,4R)-monatin)₂ calcium salt pentahydrate immediately prior to separation from a crystallization solution (200-fold magnification) (Example 1).

FIG. 3 is a powder X-ray diffraction chart of crystals of ((2R,4R)-monatin)₂ magnesium salt tetrahydrate after humidity conditioning (Example 2).

FIG. 4 is an optical microphotograph of crystals of ((2R,4R)-monatin)₂ magnesium salt tetrahydrate immediately prior to separation from a crystallization solution (200-fold magnification) (Example 2).

FIG. 5 is a powder X-ray diffraction chart of crystals of 4.6-hydrate 0.67-ethanol solvate of ((2R,4R)-monatin)₂ calcium salt after drying conditioning (Example 3).

FIG. 6 is an optical microphotograph of crystals of 4.6-hydrate 0.67-ethanol solvate of ((2R,4R)-monatin)₂ calcium salt immediately prior to separation from a crystallization solution (200-fold magnification) (Example 3).

FIG. 7 is a powder X-ray diffraction chart of crystals of 5.7-hydrate of ((2R,4R)-monatin)₂ calcium salt after drying conditioning (Example 4).

FIG. 8 is an optical microphotograph of crystals of 5.7-hydrate of ((2R,4R)-monatin)₂ calcium salt immediately prior to separation from a crystallization solution (200-fold magnification) (Example 4).

FIG. 9 is a powder X-ray diffraction chart of crystals of 5.9-hydrate 0.72-THF solvate of ((2R,4R)-monatin)₂ calcium salt after drying conditioning (Example 5).

FIG. 10 is an optical microphotograph of crystals of 5.9-hydrate 0.72-THF solvate of ((2R,4R)-monatin)₂ calcium salt immediately prior to separation from a crystallization solution (200-fold magnification) (Example 5).

FIG. 11 is a powder X-ray diffraction chart of crystals of 3.8-hydrate 0.63-i-PrOH solvate of ((2R,4R)-monatin)₂ calcium salt after drying conditioning (Example 6).

FIG. 12 is an optical microphotograph of crystals of 3.8-hydrate 0.63-i-PrOH solvate of ((2R,4R)-monatin)₂ calcium salt immediately prior to separation from a crystallization solution (200-fold magnification) (Example 6).

FIG. 13 is a powder X-ray diffraction chart of crystals of 7.5-hydrate of ((2R,4R)-monatin)₂ magnesium salt after drying (Example 7).

FIG. 14 is an optical microphotograph of crystals of 7.5-hydrate of ((2R,4R)-monatin)₂ magnesium salt immediately prior to separation from a crystallization solution (200-fold magnification) (Example 7).

FIG. 15 is a powder X-ray diffraction chart of crystals of ((2R,4R)-monatin)₂ magnesium salt dihydrate after drying (Example 8).

FIG. 16 is an optical microphotograph of crystals of ((2R,4R)-monatin)₂ magnesium salt dihydrate immediately prior to separation from a crystallization solution (200-fold magnification) (Example 8).

FIG. 17 is a powder X-ray diffraction chart of crystals of 3.1-hydrate 2.4-ethanol solvate of ((2R,4R)-monatin)₂ magnesium salt after drying (Example 9).

FIG. 18 is an optical microphotograph of crystals of 3.1-hydrate 2.4-ethanol solvate of ((2R,4R)-monatin)₂ magnesium salt immediately prior to separation from a crystallization solution (200-fold magnification) (Example 9).

FIG. 19 is a powder X-ray diffraction chart of crystals of 7.2-hydrate 0.23-methanol solvate of ((2R,4R)-monatin)₂ magnesium salt after drying (Example 10).

FIG. 20 is an optical microphotograph of crystals of 7.2-hydrate 0.23-methanol solvate of ((2R,4R)-monatin)₂ magnesium salt immediately prior to separation from a crystallization solution (200-fold magnification) (Example 10).

FIG. 21 is a powder X-ray diffraction chart of crystals of 8.5-hydrate 2.5-DMF solvate of ((2R,4R)-monatin)₂ magnesium salt after drying (Example 11).

FIG. 22 is an optical microphotograph of crystals of 8.5-hydrate 2.5-DMF solvate of ((2R,4R)-monatin)₂ magnesium salt immediately prior to separation from a crystallization solution (200-fold magnification) (Example 11).

FIG. 23 is a powder X-ray diffraction chart of crystals of ((2R,4R)-monatin)₂ magnesium salt nonahydrate after drying (Example 12).

FIG. 24 is an optical microphotograph of crystals of ((2R,4R)-monatin)₂ magnesium salt nonahydrate immediately prior to separation from a crystallization solution (200-fold magnification) (Example 12).

FIG. 25 is a water vapor adsorption-desorption curve for the crystals of ((2R,4R)-monatin)₂ magnesium salt tetrahydrate obtained by the method described in Example 2.

FIG. 26 is a water vapor adsorption-desorption curve for the crystals of ((2R,4R)-monatin)₂ magnesium salt dihydrate obtained by the method described in Example 8.

### BEST MODE FOR CARRYING OUT THE INVENTION

The present invention relates to a novel crystal of a multivalent metal salt of (2R,4R)-monatin. Monatin contains two acidic protons as shown below:

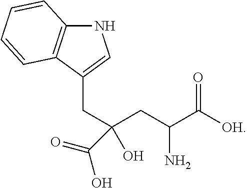 [SMILES: NC(CC(O)(Cc1c[nH]c2ccccc12)C(=O)O)C(=O)O]

Thus, the present multivalent salts are understood to have a formula in which an anion, M⁻, is formed by the removal of an acidic proton, and two or more of the anions, M⁻, form a neutral salt with a multivalent metal cation, Me^{+x}, of the formula:

(M⁻)ₓMe^{+x}

where x is both the number of M− anions in the salt and the positive charge of the multivalent metal cation, Me^{+x}.

In the present invention, the term “natural-type monatin” refers to a (2S,4S) substance in terms of its steric configuration, and all compounds having the same chemical structural formula as that of the natural-type monatin are collectively referred to as “monatin”. Accordingly, a non-natural-type stereoisomer of monatin is referred to as “a stereoisomer of natural-type monatin”, “non-natural-type monatin”, “(2S,4R)-monatin”, “(2R,4S)-monatin”, “(2R,4R)-monatin”, and the like. Further, these stereoisomers plus monatin (a (2S,4S) substance) are referred to as “four types of stereoisomers”, and particularly, the natural-type monatin is referred to as “(2S,4S)-monatin” or “(2S,4S)-monatin or the like”.

The (2R,4R)-monatin to be used in the present invention can be prepared by a known method, however, there is no restriction on the production method thereof. For example, (2R,4R)-monatin can be obtained by an enzymatic method from tryptophan through indole pyruvic acid (Patent document 4: WO 2003-056026) or can also be obtained by reduction of an oxime from tryptophan through indole pyruvic acid (Patent document 5: WO 2003-059865). It does not matter whether natural-type monatin (a (2S,4S) substance), a non-natural-type stereoisomer thereof (a (2S,4R) substance or a (2R,4S) substance) is contained in addition to (2R,4R)-monatin in the production step.

As the thus obtained (2R,4R)-monatin, a mixture containing four isomers of monatin can be used, and also monatin obtained through separation and purification using an adsorbent resin, an ion exchange resin, or other known method may be used. Further, monatin in the free form, or in the form of a known salt such as an ammonium salt, a potassium salt, or a basic amino acid salt can be temporarily adopted. A method for obtaining a multivalent metal salt of monatin is not particularly limited. However, a multivalent metal salt of monatin obtained by subjecting known monatin in the free form or in the form of a monovalent salt to neutralization or salt exchange, or a multivalent metal salt of monatin obtained through salt exchange using an ion exchange resin can be used.

The multivalent metal salt to be used in the present invention is not particularly limited as long as it is an element having two or more valence in the periodic table, the intake thereof by humans is acceptable, and it can form a salt with monatin. Specific examples thereof include divalent metal salts such as salts of alkaline earth metals (such as magnesium, calcium, strontium, and barium) and salts of transition metals (such as iron, nickel, copper, and zinc); and trivalent metal salts such as salts of metals (such as aluminum). These salts may be used alone or in combination of two or more types thereof. Among them, preferred are divalent metal salts, more preferred are alkaline earth metal salts, further more preferred are a magnesium salt, a calcium salt, a strontium salt, and a barium salt, still further more preferred are a magnesium salt, a calcium salt, and a barium salt, and particularly preferred are a magnesium salt and a calcium salt.

As a simple method for obtaining the multivalent metal salt to be used in the present invention, an inorganic multivalent metal compound such as calcium hydroxide, magnesium hydroxide, calcium carbonate, or magnesium carbonate, or an organic multivalent metal compound such as calcium acetate, magnesium acetate, calcium oxalate, magnesium oxalate, calcium lactate, or magnesium lactate can be introduced by any of a variety of methods such as neutralization or salt exchange.

Among the crystals of multivalent metal salts of (2R,4R)-monatin of the present invention, from the viewpoint that the intake thereof by humans is acceptable and the preparation thereof is easy, preferred are crystals of ((2R,4R)-monatin)₂ divalent metal salts, more preferred are crystals of ((2R,4R)-monatin)₂ alkaline earth metal salts, further more preferred are crystals of ((2R,4R)-monatin)₂ magnesium salt, crystals of ((2R,4R)-monatin)₂ calcium salt, crystals of ((2R,4R)-monatin)₂ strontium salt, and crystals of ((2R,4R)-monatin)₂ barium salt, further more preferred are crystals of ((2R,4R)-monatin)₂ magnesium salt, crystals of ((2R,4R)-monatin)₂ calcium salt, and crystals of ((2R,4R)-monatin)₂ barium salt, and particularly preferred are crystals of ((2R,4R)-monatin)₂ magnesium salt and crystals of ((2R,4R)-monatin)₂ calcium salt.

Among the crystals of multivalent metal salts of (2R,4R)-monatin of the present invention, the crystals of ((2R,4R)-monatin)₂ calcium salt will be described in detail.

An aqueous solution or an organic solvent-containing aqueous solution containing (2R,4R)-monatin obtained by the above-described method and a calcium source is subjected to crystallization by being left to stand or stirring, whereby crystals can be deposited. The concentration of the crystals of ((2R,4R)-monatin)₂ calcium salt in the solution is not particularly limited as long as supersaturation can be reached and crystals can be deposited, but the concentration thereof is preferably from 0.1 to 60 wt %. From the viewpoint of obtaining a viscosity of the solution suitable for production, the concentration thereof is more preferably from 1 to 50 wt %, further more preferably from 5 to 45 wt %. The temperature for crystallization is not particularly limited as long as the temperature allows the crystals to be deposited, but the temperature for crystallization is preferably from 15 to 100° C.

By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused.

By subjecting the thus obtained wet crystals to a known drying step, dry crystals can be obtained. A drying facility to be used in the drying step is not particularly limited, and a temperature range in which the ((2R,4R)-monatin)₂ calcium salt does not melt can be used, and vacuum drying, flush drying, hot-air drying, or the like can be used.

The ((2R,4R)-monatin)₂ calcium salt of the present invention has a polymorphism and forms significantly different crystalline forms depending on the type of crystallization solvent or the crystallization method, which will be described in detail below.

### Crystals of 4.6-hydrate 0.67-ethanol solvate of ((2R,4R)-monatin)₂ calcium salt

A method for obtaining the title crystals will be shown below.

An ethanol-containing aqueous solution containing (2R,4R)-monatin and a calcium source is subjected to crystallization by being left to stand or stirring, whereby crystals can be deposited. The concentration of the crystals of (2R,4R)-monatin in the solvent is not particularly limited as long as supersaturation can be reached and crystals can be deposited, but the concentration thereof is preferably from 1 to 60 wt %. From the viewpoint of obtaining a viscosity of the solution suitable for production, the concentration thereof is more preferably from 2 to 50 wt %, further more preferably from 5 to 45 wt %. The dissolving temperature is not particularly limited as long as the temperature allows the crystals to be continuously dissolved, but the dissolving temperature is preferably from 15 to 100° C. The ratio of ethanol in the ethanol-containing aqueous solution is from 50 to 99%, more preferably from 75 to 99%. By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused. By subjecting the thus obtained wet crystals to a known drying step, dry crystals can be obtained. A drying facility to be used in the drying step is not particularly limited, and a temperature range in which the crystals of ((2R,4R)-monatin)₂ calcium salt do not melt can be used. However, the temperature range is preferably from 10 to 60° C., and more preferably from 10 to 40° C. from the viewpoint of the stability of the quality during production, and vacuum drying, flush drying, hot-air drying, or the like can be used.

The thus obtained crystals of 4.6-hydrate 0.67-ethanol solvate of ((2R,4R)-monatin)₂ calcium salt have a needle-like crystal structure as shown in FIG. 6, and have characteristic X-ray diffraction peaks at diffraction angles (2θ±0.2°, CuKα) of 5.4°, 6.0°, 16.4°, 22.2°, and 27.3°. Further, the crystals have a property of being less likely to be degraded even when being exposed to high temperature and high humidity conditions in the coexistence of a reducing sugar than the crystals of (2R,4R)-monatin potassium salt.

### Crystals of 3.8-hydrate 0.63-isopropanol Solvate of (2R,4R)-monatin calcium Salt

A method for obtaining the title crystals will be shown below.

An isopropanol-containing aqueous solution containing (2R,4R)-monatin and a calcium source is subjected to crystallization by being left to stand or stirring, whereby crystals can be deposited. The concentration of the crystals of (2R,4R)-monatin in the solvent is not particularly limited as long as supersaturation can be reached and crystals can be deposited, but the concentration thereof is preferably from 1 to 60 wt %. From the viewpoint of obtaining a viscosity of the solution suitable for production, the concentration thereof is more preferably from 2 to 50 wt %, further more preferably from 5 to 45 wt %. The dissolving temperature is not particularly limited as long as the temperature allows the crystals to be continuously dissolved, but the dissolving temperature is preferably from 15 to 100° C. The ratio of isopropanol in the isopropanol-containing aqueous solution is from 50 to 99%, more preferably from 75 to 99%. By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused. By subjecting the thus obtained wet crystals to a known drying step, dry crystals can be obtained. A drying facility to be used in the drying step is not particularly limited, and a temperature range in which the crystals of ((2R,4R)-monatin)₂ calcium salt do not melt can be used. However, the temperature range is preferably from 10 to 60° C., and more preferably from 10 to 40° C. from the viewpoint of the stability of the quality during production, and vacuum drying, flush drying, hot-air drying, or the like can be used. The thus obtained crystals of 3.8-hydrate 0.63-isopropanol solvate of ((2R,4R)-monatin)₂ calcium salt have a needle-like crystal structure as shown in FIG. 12, and have characteristic X-ray diffraction peaks at diffraction angles (2θ±0.2°, CuKα) of 5.4°, 15.9°, 19.7°, and 22.3°. Further, the crystals have a property of being less likely to be degraded even when being exposed to high temperature and high humidity conditions in the coexistence of a reducing sugar than the crystals of (2R,4R)-monatin potassium salt.

### Crystals of 5.9-hydrate 0.72-THF solvate of ((2R,4R)-monatin)₂ calcium salt

A method for obtaining the title crystals will be shown below.

A THF-containing aqueous solution containing (2R,4R)-monatin and a calcium source is subjected to crystallization by being left to stand or stirring, whereby crystals can be deposited. The concentration of the crystals of (2R,4R)-monatin in the solvent is not particularly limited as long as supersaturation can be reached and crystals can be deposited, but the concentration thereof is preferably from 1 to 60 wt %. From the viewpoint of obtaining a viscosity of the solution suitable for production, the concentration thereof is more preferably from 2 to 50 wt %, further more preferably from 5 to 45 wt %. The dissolving temperature is not particularly limited as long as the temperature allows the crystals to be continuously dissolved, but the dissolving temperature is preferably from 15 to 100° C. The ratio of THF in the THF-containing aqueous solution is from 50 to 99%, more preferably from 75 to 99%. By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused. By subjecting the thus obtained wet crystals to a known drying step, dry crystals can be obtained. A drying facility to be used in the drying step is not particularly limited, and a temperature range in which the crystals of ((2R,4R)-monatin)₂ calcium salt do not melt can be used. However, the temperature range is preferably from 10 to 60° C., and more preferably from 10 to 40° C. from the viewpoint of the stability of the quality during production, and vacuum drying, flush drying, hot-air drying, or the like can be used.

The thus obtained crystals of 5.9-hydrate 0.72-THF solvate of ((2R,4R)-monatin)₂ calcium salt have a needle-like crystal structure as shown in FIG. 10, and have characteristic X-ray diffraction peaks at diffraction angles (2θ±0.2°, CuKα) of 5.1°, 15.9°, 19.7°, and 22.3°. Further, the crystals have a property of being less likely to be degraded even when being exposed to high temperature and high humidity conditions in the coexistence of a reducing sugar than the crystals of (2R,4R)-monatin potassium salt.

### Crystals of 5.7-hydrate of ((2R,4R)-monatin)₂ calcium salt

A method for obtaining the title crystals will be shown below.

An aqueous solution containing (2R,4R)-monatin and a calcium source is subjected to crystallization by being left to stand or stirring, whereby crystals can be deposited. However, particularly, an acetonitrile-containing aqueous solution is preferred. The concentration of the crystals of (2R,4R)-monatin in the solvent is not particularly limited as long as supersaturation can be reached and crystals can be deposited, but the concentration thereof is preferably from 1 to 60 wt %. From the viewpoint of obtaining a viscosity of the solution suitable for production, the concentration thereof is more preferably from 2 to 50 wt %, further more preferably from 5 to 45 wt %. The dissolving temperature is not particularly limited as long as the temperature allows the crystals to be continuously dissolved, but the dissolving temperature is preferably from 15 to 100° C. In the case of using an acetonitrile-containing aqueous solution, the ratio of acetonitrile in the acetonitrile-containing aqueous solution is from 50 to 99%, more preferably from 75 to 99%. By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused. By subjecting the thus obtained wet crystals to a known drying step, dry crystals can be obtained. A drying facility to be used in the drying step is not particularly limited, and a temperature range in which the crystals of ((2R,4R)-monatin)₂ calcium salt do not melt can be used. However, the temperature range is preferably from 10 to 60° C., and more preferably from 10 to 40° C. from the viewpoint of the stability of the quality during production, and vacuum drying, flush drying, hot-air drying, or the like can be used.

The thus obtained crystals of 5.7-hydrate of ((2R,4R)-monatin)₂ calcium salt have a needle-like crystal structure as shown in FIG. 8, and have characteristic X-ray diffraction peaks at diffraction angles (2θ±0.2°, CuKα) of 5.0°, 12.8°, 15.3°, 18.1°, and 23.7°. Further, the crystals have a property of being less likely to be degraded even when being exposed to high temperature and high humidity conditions in the coexistence of a reducing sugar than the crystals of (2R,4R)-monatin potassium salt.

### Crystals of ((2R,4R)-monatin)₂ calcium salt pentahydrate

A method for obtaining the title crystals will be shown below.

An organic solvent-containing aqueous solution containing (2R,4R)-monatin and a calcium source is subjected to crystallization by being left to stand or stirring, whereby crystals can be deposited. The concentration of the crystals of (2R,4R)-monatin in the solvent is not particularly limited as long as supersaturation can be reached and crystals can be deposited, but the concentration thereof is preferably from 1 to 60 wt %. From the viewpoint of obtaining a viscosity of the solution suitable for production, the concentration thereof is more preferably from 2 to 50 wt %, further more preferably from 5 to 45 wt %. The dissolving temperature is not particularly limited as long as the temperature allows the crystals to be continuously dissolved, but the dissolving temperature is preferably from 15 to 100° C. The type of the organic solvent in the organic solvent-containing aqueous solution is not particularly limited, but preferred is a water-soluble organic solvent having a boiling point of 100° C. or lower, and more preferred is a water-soluble organic solvent having a boiling point of 80° C. or lower. The ratio of the organic solvent therein is from 50 to 99%, more preferably from 75 to 99%. Examples of the organic solvent to be used include solvents miscible with water such as methanol, ethanol, n-propanol, isopropanol, n-butanol, t-butanol, sec-butanol, propylene glycol, and THF. Examples of the preferred solvent include ethanol. By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused. By subjecting the thus obtained wet crystals to a known drying step, dry crystals can be obtained. A drying facility to be used in the drying step is not particularly limited, and a temperature range in which the crystals of ((2R,4R)-monatin)₂ calcium salt do not melt can be used. However, the temperature range is preferably from 10 to 60° C., and more preferably from 10 to 40° C. from the viewpoint of the stability of the quality during production, and vacuum drying, flush drying, hot-air drying, or the like can be used. Further, in the case where the crystals are in the form of an organic solvate, by storing the crystals under high temperature and high humidity conditions, desired crystals can be obtained. As for the temperature at this time, the crystals are stored at a temperature of from 25 to 100° C., more preferably from 40 to 80° C. As for the relative humidity range, the crystals are stored at a relative humidity of from 20 to 100%, more preferably from 60 to 100%. As for the storage time, the crystals are stored for 24 to 168 hours, more preferably for 48 to 120 hours.

The thus obtained crystals of ((2R,4R)-monatin)₂ calcium salt pentahydrate have a needle-like crystal structure as shown in FIG. 2, and have characteristic X-ray diffraction peaks at diffraction angles (2θ±0.2°, CuKα) of 6.0°, 9.8°, 16.0°, 21.5° and 22.3°. Further, the crystals have a property of being less likely to be degraded even when being exposed to high temperature and high humidity conditions in the coexistence of a reducing sugar than the crystals of (2R,4R)-monatin potassium salt. Moreover, the crystals have a property of being less likely to be degraded even when being exposed to high temperature and high humidity conditions in the coexistence of a reducing sugar than the crystals of organic solvent solvates of ((2R,4R)-monatin)₂ calcium salt.

Among the crystals of ((2R,4R)-monatin)₂ calcium salts, from the viewpoint of being stable even in the coexistence of a reducing sugar under high temperature and high humidity conditions, preferred are crystals of 4.6-hydrate 0.67-ethanol solvate of ((2R,4R)-monatin)₂ calcium salt, crystals of 3.8-hydrate 0.63-isopropanol solvate of ((2R,4R)-monatin)₂ calcium salt, crystals of 5.9-hydrate 0.72-THF solvate of ((2R,4R)-monatin)₂ calcium salt, crystals of 5.7-hydrate of ((2R,4R)-monatin)₂ calcium salt, and crystals of ((2R,4R)-monatin)₂ calcium salt pentahydrate, more preferred are crystals of 5.9-hydrate 0.72-THF solvate of ((2R,4R)-monatin)₂ calcium salt, crystals of 5.7-hydrate of ((2R,4R)-monatin)₂ calcium salt, and crystals of ((2R,4R)-monatin)₂ calcium salt pentahydrate, further more preferred are crystals of 5.7-hydrate of ((2R,4R)-monatin)₂ calcium salt and crystals of ((2R,4R)-monatin)₂ calcium salt pentahydrate, and particularly preferred are crystals of ((2R,4R)-monatin)₂ calcium salt pentahydrate.

Subsequently, among the crystals of multivalent metal salts of (2R,4R)-monatin of the present invention, the crystals of ((2R,4R)-monatin)₂ magnesium salt will be described in detail.

An aqueous solution or an organic solvent-containing aqueous solution containing (2R,4R)-monatin obtained by the above-described method and a magnesium source is subjected to crystallization by being left to stand or stirring, whereby crystals can be deposited. The concentration of the crystals of ((2R,4R)-monatin)₂ magnesium salt in the solution is not particularly limited as long as supersaturation can be reached and crystals can be deposited, but, the concentration thereof is preferably from 0.1 to 60 wt %. From the viewpoint of obtaining a viscosity of the solution suitable for production, the concentration thereof is more preferably from 1 to 50 wt %, furthermore preferably from 5 to 45 wt %. The temperature for crystallization is not particularly limited as long as the temperature allows the crystals to be deposited, but the temperature for crystallization is preferably from 15 to 100° C.

By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused.

By subjecting the thus obtained wet crystals to a known drying step, dry crystals can be obtained. A drying facility to be used in the drying step is not particularly limited, and a temperature range in which the ((2R,4R)-monatin)₂ magnesium salt does not melt can be used, and vacuum drying, flush drying, hot-air drying, or the like can be used.

The ((2R,4R)-monatin)₂ magnesium salt of the present invention has a polymorphism and forms significantly different crystalline forms depending on the type of crystallization solvent or the crystallization method, which will be described in detail below.

### Crystals of 3.1-hydrate 2.4-ethanol solvate of ((2R,4R)-monatin)₂ magnesium salt

A method for obtaining the title crystals will be shown below.

An ethanol-containing aqueous solution containing (2R,4R)-monatin and a magnesium source is subjected to crystallization by being left to stand or stirring, whereby crystals can be deposited. The concentration of the crystals of (2R,4R)-monatin in the solvent is not particularly limited as long as supersaturation can be reached and crystals can be deposited, but the concentration thereof is preferably from 1 to 60 wt %. From the viewpoint of obtaining a viscosity of the solution suitable for production, the concentration thereof is more preferably from 2 to 50 wt %, further more preferably from 5 to 45 wt %. The dissolving temperature is not particularly limited as long as the temperature allows the crystals to be continuously dissolved, but the dissolving temperature is preferably from 15 to 100° C. The ratio of ethanol in the ethanol-containing aqueous solution is from 50 to 99%, more preferably from 75 to 99%. By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused. By subjecting the thus obtained wet crystals to a known drying step, dry crystals can be obtained. A drying facility to be used in the drying step is not particularly limited, and a temperature range in which the crystals of ((2R,4R)-monatin)₂ magnesium salt do not melt can be used. However, the temperature range is preferably from 10 to 60° C., and more preferably from 10 to 40° C. from the viewpoint of the stability of the quality during production, and vacuum drying, flush drying, or the like can be used.

The thus obtained crystals of 3.1-hydrate 2.4-ethanol solvate of ((2R,4R)-monatin)₂ magnesium salt have a microcrystalline structure as shown in FIG. 18, and have characteristic X-ray diffraction peaks at diffraction angles (2θ±0.2°, CuKα) of 7.2°, 10.0°, 10.6°, 12.3°, 14.8°, 17.8°, and 25.3°. Further, the crystals have a property of being less likely to be degraded even when being exposed to high temperature and high humidity conditions in the coexistence of a reducing sugar than the crystals of (2R,4R)-monatin potassium salt.

### Crystals of 7.2-hydrate 0.23-methanol solvate of ((2R,4R)-monatin)₂ magnesium salt

A method for obtaining the title crystals will be shown below.

A methanol-containing aqueous solution containing (2R,4R)-monatin and a magnesium source is subjected to crystallization by being left to stand or stirring, whereby crystals can be deposited. The concentration of the crystals of (2R,4R)-monatin in the solvent is not particularly limited as long as supersaturation can be reached and crystals can be deposited, but the concentration thereof is preferably from 1 to 60 wt %. From the viewpoint of obtaining a viscosity of the solution suitable for production, the concentration thereof is more preferably from 2 to 50 wt %, further more preferably from 5 to 45 wt %. The dissolving temperature is not particularly limited as long as the temperature allows the crystals to be continuously dissolved, but the dissolving temperature is preferably from 15 to 100° C. The ratio of methanol in the methanol-containing aqueous solution is from 50 to 99%, more preferably from 75 to 99%. By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused. By subjecting the thus obtained wet crystals to a known drying step, dry crystals can be obtained. A drying facility to be used in the drying step is not particularly limited, and a temperature range in which the crystals of ((2R,4R)-monatin)₂ magnesium salt do not melt can be used. However, the temperature range is preferably from 10 to 60° C., and more preferably from 10 to 40° C. from the viewpoint of the stability of the quality during production, and vacuum drying, flush drying, or the like can be used.

The thus obtained crystals of 7.2-hydrate 0.23-methanol solvate of ((2R,4R)-monatin)₂ magnesium salt have a microcrystalline structure as shown in FIG. 20, and have characteristic X-ray diffraction peaks at diffraction angles (2θ±0.2°, CuKα) of 8.0°, 10.0°, 10.3°, 11.4°, 16.1°, 19.0°, and 23.7°. Further, the crystals have a property of being less likely to be degraded even when being exposed to high temperature and high humidity conditions in the coexistence of a reducing sugar than the crystals of (2R,4R)-monatin potassium salt.

### Crystals of 8.5-hydrate 2.5-DMF solvate of ((2R,4R)-monatin)₂ magnesium salt

A method for obtaining the title crystals will be shown below.

A DMF-containing aqueous solution containing (2R,4R)-monatin and a magnesium source is subjected to crystallization by being left to stand or stirring, whereby crystals can be deposited. The concentration of the crystals of (2R,4R)-monatin in the solvent is not particularly limited as long as supersaturation can be reached and crystals can be deposited, but the concentration thereof is preferably from 1 to 60 wt %. From the viewpoint of obtaining a viscosity of the solution suitable for production, the concentration thereof is more preferably from 2 to 50 wt %, further more preferably from 5 to 45 wt %. The dissolving temperature is not particularly limited as long as the temperature allows the crystals to be continuously dissolved, but the dissolving temperature is preferably from 15 to 100° C. The ratio of DMF in the DMF-containing aqueous solution is from 50 to 99%, more preferably from 75 to 99%. By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused. By subjecting the thus obtained wet crystals to a known drying step, dry crystals can be obtained. A drying facility to be used in the drying step is not particularly limited, and a temperature range in which the crystals of ((2R,4R)-monatin)₂ magnesium salt do not melt can be used. However, the temperature range is preferably from 10 to 60° C., and more preferably from 10 to 40° C. from the viewpoint of the stability of the quality during production, and vacuum drying, flush drying, or the like can be used.

The thus obtained crystals of 8.5-hydrate 2.5-DMF solvate of ((2R,4R)-monatin)₂ magnesium salt have a microcrystalline structure as shown in FIG. 22, and have characteristic X-ray diffraction peaks at diffraction angles (2θ±0.2°, CuKα) of 7.5°, 10.3°, 11.2°, 16.0°, 18.1°, and 23.0°. Further, the crystals have a property of being less likely to be degraded even when being exposed to high temperature and high humidity conditions in the coexistence of a reducing sugar than the crystals of (2R,4R)-monatin potassium salt.

### Crystals of ((2R,4R)-monatin)₂ magnesium salt nonahydrate

A method for obtaining the title crystals will be shown below.

An aqueous solution or an organic solvent-containing aqueous solution containing (2R,4R)-monatin and a magnesium source is subjected to crystallization by being left to stand or stirring, whereby crystals can be deposited. The concentration of the crystals of (2R,4R)-monatin in the solvent is not particularly limited as long as supersaturation can be reached and crystals can be deposited, but the concentration thereof is preferably from 1 to 60 wt %. From the viewpoint of obtaining a viscosity of the solution suitable for production, the concentration thereof is more preferably from 2 to 50 wt %, further more preferably from 5 to 45 wt %. The dissolving temperature is not particularly limited as long as the temperature allows the crystals to be continuously dissolved, but the dissolving temperature is preferably from 10 to 100° C. The temperature of a slurry containing the deposited crystals is preferably from 10 to 100° C., more preferably from 10 to 65° C. If the temperature of the slurry solution is 65° C. or higher, the time of keeping the slurry solution is 24 hours or less, and if the temperature of the slurry solution is 65° C. or lower, the time of keeping the slurry solution is not particularly limited. Further, in order to obtain target crystals stably at 65° C. or higher, the concentration of inorganic anions is preferably 0.028 N/kg or less, more preferably 0.0069 N/kg or less. The term “the concentration of inorganic anions” as used herein refers to the normality (N) of the concentration of salts with respect to the total weight (kg).

By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused.

By subjecting the thus obtained wet crystals to controlled drying conditions, dry crystals can be obtained. A temperature range in which the crystals of ((2R,4R)-monatin)₂ magnesium salt do not melt can be used, but the temperature range is preferably from 10 to 60° C., and more preferably from 10 to 40° C. from the viewpoint of the stability of the quality during production. As the drying time, an arbitrary time can be selected as long as the crystals are not overdried. However, the drying time is preferably 6 hours or less, and more preferably 4 hours or less from the viewpoint of the stability of the water content, and vacuum drying, flush drying, or the like can be used.

The thus obtained crystals of ((2R,4R)-monatin)₂ magnesium salt nonahydrate have a columnar crystal structure as shown in FIG. 24, and have characteristic X-ray diffraction peaks at diffraction angles (2θ±0.2°, CuKα) of 8.7°, 10.5°, 15.9°, 17.4°, 21.0°, and 25.6°. Further, the crystals have a property of being less likely to be degraded even when being exposed to high temperature and high humidity conditions in the coexistence of a reducing sugar than the crystals of (2R,4R)-monatin potassium salt. Moreover, the crystals have a property of being less likely to be degraded even when being exposed to high temperature and high humidity conditions in the coexistence of a reducing sugar than the crystals of organic solvent solvates of ((2R,4R)-monatin)₂ magnesium salt.

### Crystals of 7.5-hydrate of ((2R,4R)-monatin)₂ magnesium salt

A method for obtaining the title crystals will be shown below.

An aqueous solution or an organic solvent-containing aqueous solution containing (2R,4R)-monatin and a magnesium source is subjected to crystallization by being left to stand or stirring, whereby crystals can be deposited. The concentration of the crystals of (2R,4R)-monatin in the solvent is not particularly limited as long as supersaturation can be reached and crystals can be deposited, but the concentration thereof is preferably from 1 to 60 wt %. From the viewpoint of obtaining a viscosity of the solution suitable for production, the concentration thereof is more preferably from 2 to 50 wt %, further more preferably from 5 to 45 wt %. The dissolving temperature is not particularly limited as long as the temperature allows the crystals to be continuously dissolved, but the dissolving temperature is preferably from 10 to 100° C. The temperature of a slurry containing the deposited crystals is preferably from 10 to 100° C., more preferably from 10 to 65° C. If the temperature of the slurry solution is 65° C. or higher, the time of keeping the slurry solution is 24 hours or less, and if the temperature of the slurry solution is 65° C. or lower, the time of keeping the slurry solution is not particularly limited. Further, in order to obtain target crystals stably at 65° C. or higher, the concentration of inorganic anions is preferably 0.028 N/kg or less, more preferably 0.0069 N/kg or less. The term “the concentration of inorganic anions” as used herein refers to the normality (N) of the concentration of salts with respect to the total weight (kg).

By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused.

By subjecting the thus obtained wet crystals to controlled drying conditions at a low temperature, dry crystals can be obtained. A temperature range in which the crystals of ((2R,4R)-monatin)₂ magnesium salt do not melt can be used, but the temperature range is preferably from 10 to 60° C., and more preferably from 10 to 40° C. from the viewpoint of the stability of the quality during production. As the drying time, an arbitrary time can be selected as long as the crystals are not overdried, and vacuum drying, flush drying, or the like can be used.

The thus obtained crystals of 7.5-hydrate of ((2R,4R)-monatin)₂ magnesium salt have a columnar crystal structure as shown in FIG. 14, and have characteristic X-ray diffraction peaks at diffraction angles (2θ±0.2°, CuKα) of 8.7°, 10.5°, 15.9°, 17.4°, 21.0°, and 25.6°. Further, the crystals have a property of being less likely to be degraded even when being exposed to high temperature and high humidity conditions in the coexistence of a reducing sugar than the crystals of (2R,4R)-monatin potassium salt. Moreover, the crystals have a property of being less likely to be degraded even when being exposed to high temperature and high humidity conditions in the coexistence of a reducing sugar than the crystals of organic solvent solvates of ((2R,4R)-monatin)₂ magnesium salt.

### Crystals of ((2R,4R)-monatin)₂ magnesium salt tetrahydrate

A method for obtaining the title crystals will be shown below.

An organic solvent-containing aqueous solution containing (2R,4R)-monatin and a magnesium source is subjected to crystallization by being left to stand or stirring, whereby crystals can be deposited. The concentration of the crystals of (2R,4R)-monatin in the solvent is not particularly limited as long as supersaturation can be reached and crystals can be deposited, but the concentration thereof is preferably from 1 to 60 wt %. From the viewpoint of obtaining a viscosity of the solution suitable for production, the concentration thereof is more preferably from 2 to 50 wt %, further more preferably from 5 to 45 wt %. The dissolving temperature is not particularly limited as long as the temperature allows the crystals to be continuously dissolved, but the dissolving temperature is preferably from 15 to 100° C. The type of the organic solvent in the organic solvent-containing aqueous solution is not particularly limited, however, preferred is a water-soluble organic solvent having a boiling point of 100° C. or lower, and more preferred is a water-soluble organic solvent having a boiling point of 80° C. or lower. The ratio of the organic solvent therein is from 50 to 99%, more preferably from 75 to 99%. By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused. By subjecting the thus obtained wet crystals to controlled drying conditions, dry crystals can be obtained. A drying facility to be used in the drying step is not particularly limited, and a temperature range in which the crystals of ((2R,4R)-monatin)₂ magnesium salt do not melt can be used. However, the temperature range is preferably from 25 to 120° C., and more preferably from 40 to 100° C. from the viewpoint of the stability of the quality during production, and vacuum drying, flush drying, hot-air drying, or the like can be used. Further, in the case where the crystals are in the form of an organic solvate, by storing the crystals under high temperature and high humidity conditions, desired crystals can be obtained. As for the temperature at this time, the crystals are stored at a temperature of from 25 to 100° C., more preferably from 40 to 80° C. As for the relative humidity range, the crystals are stored at a relative humidity of from 20 to 100%, more preferably from 60 to 100%. As for the storage time, the crystals are stored for 24 to 168 hours, more preferably for 48 to 120 hours.

The thus obtained crystals of ((2R,4R)-monatin)₂ magnesium salt tetrahydrate have a microcrystalline structure as shown in FIG. 4, and have characteristic X-ray diffraction peaks at diffraction angles (2θ±0.2°, CuKα) of 8.9°, 11.2°, 15.0°, 17.8°, and 22.5°. Further, the crystals have a property of being less likely to be degraded even when being exposed to high temperature and high humidity conditions in the coexistence of a reducing sugar than the crystals of (2R,4R)-monatin potassium salt.

### Crystals of ((2R,4R)-monatin)₂ magnesium salt dihydrate

A method for obtaining the title crystals will be shown below.

An aqueous solution or an organic solvent-containing aqueous solution containing (2R,4R)-monatin and a magnesium source is subjected to crystallization by being left to stand or stirring, whereby crystals can be deposited. The concentration of the crystals of (2R,4R)-monatin in the solvent is not particularly limited as long as supersaturation can be reached and crystals can be deposited, but the concentration thereof is preferably from 1 to 60 wt %. From the viewpoint of obtaining a viscosity of the solution suitable for production, the concentration thereof is more preferably from 2 to 50 wt %, further more preferably from 5 to 45 wt %. The dissolving temperature is not particularly limited as long as the temperature allows the crystals to be continuously dissolved, but the dissolving temperature is preferably from 10 to 100° C. The temperature of a slurry containing the deposited crystals is preferably from 10 to 100° C., more preferably from 65 to 100° C. The time of keeping the slurry solution is not particularly limited. The target crystals can be obtained by means of a high salting-out effect whether the crystals are deposited at a high temperature or a low temperature. In the case where the target crystals are obtained by only controlling the temperature, the crystallization temperature is preferably 50° C. or higher, more preferably 55° C. or higher, further more preferably 60° C. or higher, and particularly preferably 65° C. or higher. Further, in the case where the target crystals are obtained stably at lower than 65° C., the concentration of inorganic anions is preferably 0.14 N/kg or more, more preferably 0.28 N/kg or more, further more preferably 0.55 N/kg or more, and particularly preferably 0.88 N/kg or more. The term “the concentration of inorganic anions” as used herein refers to the normality (N) of the concentration of salts with respect to the total weight (kg).

The ratio of the organic solvent in the organic solvent-containing aqueous solution is from 0.1 to 75%, more preferably from 0.1 to 50%.

By subjecting the deposited crystals to a separation step such as a filtration step, wet crystals can be easily obtained. A solvent to be used when washing the crystals is not particularly limited as long as crystal solvent exchange is not caused, but water can be used. Further, a solvent miscible with water such as methanol, ethanol, n-propanol, isopropanol, n-butanol, t-butanol, sec-butanol, propylene glycol, acetonitrile, or THF, or an inorganic salt or the like may be contained in water as long as crystal solvent exchange is not caused.

By subjecting the thus obtained wet crystals to a known drying step, dry crystals can be obtained. A drying facility to be used in the drying step is not particularly limited, and a temperature range in which the crystals of ((2R,4R)-monatin)₂ magnesium salt do not melt can be used. However, the temperature range is preferably from 10 to 120° C., and more preferably from 60 to 120° C. from the viewpoint of the stability of the quality during production, and vacuum drying, flush drying, or the like can be used.

The thus obtained crystals of ((2R,4R)-monatin)₂ magnesium salt dihydrate have a needle-like crystal structure as shown in FIG. 16, and have characteristic X-ray diffraction peaks at diffraction angles (2θ±0.2°, CuKα) of 4.9°, 16.8°, 18.0°, and 24.6°. Further, the crystals have a property of being less likely to be degraded even when being exposed to high temperature and high humidity conditions in the coexistence of a reducing sugar than the crystals of (2R,4R)-monatin potassium salt. Moreover, the crystals have a property of being less likely to be degraded even when being exposed to high temperature and high humidity conditions in the coexistence of a reducing sugar than the crystals of organic solvent solvates of ((2R,4R)-monatin)₂ magnesium salt. Still moreover, the crystals have a property of being less likely to be degraded even when being exposed to high temperature and high humidity conditions in the coexistence of a reducing sugar than the crystals of ((2R,4R)-monatin)₂ magnesium salt tetrahydrate or the crystals of 7.5 hydrate of ((2R,4R)-monatin)₂ magnesium salt or the crystals of ((2R,4R)-monatin)₂ magnesium salt nonahydrate, and therefore are most useful.

Among the crystals of ((2R,4R)-monatin)₂ magnesium salts, from the viewpoint of being stable even in the coexistence of a reducing sugar under high temperature and high humidity conditions, preferred are crystals of 3.1-hydrate 2.4-ethanol solvate of ((2R,4R)-monatin)₂ magnesium salt, crystals of 7.2-hydrate 0.23-methanol solvate of ((2R,4R)-monatin)₂ magnesium salt, crystals of 8.5-hydrate 2.5-DMF solvate of ((2R,4R)-monatin)₂ magnesium salt, crystals of ((2R,4R)-monatin)₂ magnesium salt nonahydrate, crystals of 7.5-hydrate of ((2R,4R)-monatin)₂ magnesium salt, crystals of ((2R,4R)-monatin)₂ magnesium salt tetrahydrate, and crystals of ((2R,4R)-monatin)₂ magnesium salt dihydrate, more preferred are crystals of 8.5-hydrate 2.5-DMF solvate of ((2R,4R)-monatin)₂ magnesium salt, crystals of ((2R,4R)-monatin)₂ magnesium salt nonahydrate, crystals of 7.5-hydrate of ((2R,4R)-monatin)₂ magnesium salt, crystals of ((2R,4R)-monatin)₂ magnesium salt tetrahydrate, and crystals of ((2R,4R)-monatin)₂ magnesium salt dihydrate, further more preferred are crystals of ((2R,4R)-monatin)₂ magnesium salt nonahydrate, crystals of 7.5-hydrate of ((2R,4R)-monatin)₂ magnesium salt, crystals of ((2R,4R)-monatin)₂ magnesium salt tetrahydrate, and crystals of ((2R,4R)-monatin)₂ magnesium salt dihydrate, still further more preferred are crystals of 7.5-hydrate of ((2R,4R)-monatin)₂ magnesium salt, crystals of ((2R,4R)-monatin)₂ magnesium salt tetrahydrate, and crystals of ((2R,4R)-monatin)₂ magnesium salt dihydrate, yet still further more preferred are crystals of ((2R,4R)-monatin)₂ magnesium salt tetrahydrate and crystals of ((2R,4R)-monatin)₂ magnesium salt dihydrate, and particularly preferred are crystals of ((2R,4R)-monatin)₂ magnesium salt dihydrate.

Incidentally, the crystals of multivalent metal salts of (2R,4R)-monatin of the present invention should be considered to be the same crystals as long as the crystals have the same set of diffraction peaks as shown in this specification even if the ratio between monatin and the metal, water, or the solvent varies slightly.

The crystals of multivalent metal salts of (2R,4R)-monatin of the present invention can further form monatin crystals together with crystals of multivalent metal salts of (2S,4S)-monatin as another isomer of monatin. The enantiomeric excess thereof in this case is not particularly limited, but from the viewpoint of maintaining stable crystals and exhibiting an effective quality of sweetness in a small amount, the enantiomeric excess thereof is preferably from 10 to 100% ee, more preferably from 30 to 100% ee, further more preferably from 50 to 100% ee, still further more preferably from 70 to 100% ee, yet still further more preferably from 90 to 100% ee, and particularly preferably from 95 to 100% ee.

The crystals of multivalent metal salts of (2R,4R)-monatin of the present invention can further form monatin crystals together with crystals of multivalent metal salts of (2S,4R)-monatin or crystals of multivalent metal salts of (2R,4S)-monatin as another isomer of monatin. The diastereomeric excess thereof is not particularly limited, however, from the viewpoint of maintaining stable crystals and exhibiting an effective quality of sweetness in a small amount, the diastereomeric excess thereof is preferably from 10 to 100% de, more preferably from 30 to 100% de, further more preferably from 50 to 100% de, still further more preferably from 70 to 100% de, yet still further more preferably from 90 to 100% de, and particularly preferably from 95 to 100% de.

The crystals of multivalent metal salts of (2R,4R)-monatin of the present invention can further form monatin crystals together with another inorganic or organic impurity. The lower limit of chemical purity of the monatin crystals containing the crystals of multivalent metal salts of (2R,4R)-monatin of the present invention is not particularly limited as long as the crystals can be formed, but from the viewpoint that stable crystals can be formed, the lower limit thereof is preferably 50% by mass, more preferably 60% by mass, further more preferably 70% by mass, still further more preferably 80% by mass, yet still further more preferably 90% by mass, and particularly preferably 95% by mass. On the other hand, the upper limit of chemical purity thereof is preferably 100% by mass from the viewpoint of achieving a sweetness intensity even in a smaller amount. The “chemical purity” as used herein is the ratio of the mass of the “crystals of a multivalent metal salt hydrate of monatin” to the total mass of the monatin crystals. Examples of a cause of decreasing the purity include impurities (including other isomers) in the monatin itself, inorganic salts, and salts of metals other than calcium and magnesium. However, the cause is not limited thereto.

The crystals of multivalent metal salts of (2R,4R)-monatin of the present invention can further form monatin crystals together with a multivalent metal salt of (2S,4S)-monatin, a multivalent metal salt of (2S,4R)-monatin, or a multivalent metal salt of (2R,4S)-monatin as another isomer of monatin, or another inorganic or organic impurity. The sweetness intensity of the monatin crystals containing the crystals of multivalent metal salts of (2R,4R)-monatin of the present invention is not particularly limited, however, from the viewpoint of maintaining stable crystals and exhibiting an effective quality of sweetness in a small amount, the sweetness intensity thereof is higher than that of an aqueous solution of 5% sucrose by preferably 200 times or more, more preferably 500 times or more, further more preferably 1000 times or more, still further more preferably 1500 times or more, yet still further more preferably 2000 times or more, and particularly preferably 2500 times or more.

The crystals of multivalent metal salts of (2R,4R)-monatin of the present invention can be widely used as a sweetener composition. The form of the sweetener composition is not particularly limited, but examples thereof include a liquid, a powder, and a solid. In particular, from the viewpoint that a stabilizing effect derived from the crystal structure can be sufficiently exhibited, a powder and a solid are preferred, and a powder is particularly preferred.

The sweetener composition of the present invention may further contain a reducing sugar. This sweetener composition has a property that monatin is less likely to be degraded even when being exposed to high temperature and high humidity conditions.

The reducing sugar to be used in the present invention is not particularly limited as long as it is a sugar that has a reducing ability and can cause a Maillard reaction. Specific examples thereof include monosaccharides such as dihydroxyacetone, glyceraldehyde, erythrulose, erythrose, threose, ribulose, xylulose, ribose, arabinose, xylose, lyxose, deoxyribose, psicose, fructose, sorbose, tagatose, allose, altrose, glucose, mannose, gulose, idose, galactose, talose, fucose, fucrose, rhamnose, and sedoheptulose; disaccharides such as lactose, maltose, turanose, sucrose, trehalose, and llobiose. From the viewpoint that the sweetness characteristic is favorable and the needs from the market are high, preferred are glucose, fructose, maltose, lactose, galactose, mannose, arabinose, and xylose, more preferred are glucose, fructose, maltose, and lactose, and further more preferred are glucose and maltose.

Further, the reducing sugar to be used in the present invention can be substituted by a “substance that can produce a reducing sugar in the formulation”, that is, a reducing sugar-producing substance. The reducing sugar-producing substance of the present invention is not particularly limited as long as it can produce a reducing sugar according to the respective formulation conditions. Specific examples thereof include sucrose and trehalose. From the viewpoint that the sweetness characteristic is favorable and the needs from the market are high, sucrose is preferred.

Conventionally, in the case where a sweetener composition containing known monatin crystals (such as (2R,4R)-monatin monopotassium salt or (2R,4R)-monatin monosodium salt) and a reducing sugar was prepared, a phenomenon that monatin was liable to disappear under high temperature and high humidity conditions was observed. However, by using the crystals of multivalent metal salts of (2R,4R)-monatin of the present invention, such a phenomenon could be significantly improved, which is very significant.

It is presumed that the cause of the disappearance of monatin is a Maillard reaction between the reducing sugar and the amino group of monatin. It is considered that since a plurality of monatin molecules are bonded to the multivalent metal, the crystals may possibly have a structure that the amino group of monatin is sterically covered to prevent the reducing sugar from coming close to the amino group.

In the sweetener composition of the present invention, another sweetener (except for monatin or a salt thereof) can be further blended. The another sweetener is not particularly limited, but specific examples thereof include oligosaccharides such as fructooligosaccharide, maltooligosaccharide, isomaltooligosaccharide, and galactooligosaccharide; sugar alcohols such as xylitol, lactitol, sorbitol, erythritol, mannitol, maltitol, reduced palatinose, and reduced starch saccharification products; and high-intensity sweeteners (HIS) such as aspartame, acesulfame-K, sucralose, saccharin, stevioside, neotame, sodium cyclohexyl sulfamate, stevia, glycyrrhizin, monellin, thaumatin, alitame, dulcin, brazzein, neoculin, and MHPPAPM (N—[N-[3-(3-hydroxy-4-methoxyphenyl)propyl]-L-α-aspartyl]-L-phenylalanine 1-methylester monohydrate (Advantame (CAS No. 714229-20-6)). These may be used alone or in admixture of two or more thereof. From the viewpoint of achieving a synergistic sweetening effect, preferred are aspartame, acesulfame-K, sucralose, saccharin, sodium cyclohexyl sulfamate, stevioside, and neotame, more preferred are aspartame, acesulfame-K, sucralose, saccharin, stevioside, and neotame, further more preferred are aspartame, acesulfame-K, sucralose, stevioside, and neotame, still further more preferred are aspartame, acesulfame-K, sucralose, and neotome, yet still further more preferred are aspartame, acesulfame-K, and sucralose, and particularly preferred are aspartame and sucralose. From the viewpoint of the quality of taste and achieving a synergistic sweetening effect, aspartame is particular preferred.

In addition to a variety of food materials, a variety of additives that can be used in oral products such as drinks, foods, pharmaceuticals, quasi drugs, and feeds can be used to such an extent that the effect of the present invention is not inhibited. Specific examples thereof include excipients such as dextrins (such as dextrin, maltodextrin, starch decomposition products, reduced starch decomposition products, cyclodextrin, and resistant dextrins) and polysaccharides (such as crystalline cellulose and polydextrose); pH adjusting agents such as citric acid, phosphoric acid, lactic acid, malic acid, tartaric acid, gluconic acid, and salts thereof; antioxidants such as L-ascorbic acid, erysorbic acid, and tocopherol (vitamin E); shelf life-prolonging agents such as sodium acetate, glycine, glycerine fatty acid esters, and lysozyme; preservatives such as sodium benzoate and potassium sorbate; stabilizers such as pectin, gum arabic, carageenan, soy polysaccharides, and hydroxypropyl cellulose (HPC); thickening stabilizers such as xanthan gum, locust bean gum, guar gum, tamarind gum, and caraya gum; anticaking agents such as calcium phosphate, calcium carbonate, magnesium carbonate, silicon dioxide, and shell calcium; fragrances such as natural fragrances (such as cinnamon oil, lemon oil, mint oil, orange oil, and vanilla), synthetic fragrances (such as menthol, citral, cinnamic alcohol, terpineol, and vanillin), and mixed fragrances obtained by mixing the same; coloring materials such as gardenia dye, caramel dye, cochineal dye, annatto dye, safflower dye, β-carotene, and a variety of tar-based synthetic dyes; disintegrants such as sodium hydrogen carbonate, starch, agar powder, gelatin powder, and crystalline cellulose; lubricants such as stearic acid, sugar esters, benzoic acid, and talc; leavening agents such as sodium hydrogen carbonate and glucono delta-lactone; and emulsifying agents such as lecithin, sucrose fatty acid esters, glycerine fatty acid esters, and sorbitan fatty acid esters. These additives can be used in combination at an arbitrary ratio, and these additives may be used alone or in admixture of two or more thereof.

The crystal of a multivalent metal salt of monatin or the sweetener composition of the present invention can be used in oral products such as drinks, foods, pharmaceuticals, quasi drugs, and feeds. The form of such a product is not particularly limited, but examples thereof include a powder, a granule, a cube, a paste, and a liquid. Specific examples thereof include drinks typified by liquid drinks such as fruit drinks, vegetable drinks, cola, carbonated drinks, sports drinks, coffee, black tea, cocoa, and milk-based drinks, powdered drinks such as powdered juices, and liquors such as plum liqueur, medicinal liqueur, fruit liqueur, and sake; foods typified by confectionery such as chocolate, cookies, cakes, doughnuts, chewing gum, jelly, pudding, mousse, and Japanese-style confectionery, breads such as French bread and croissants, dairy products such as coffee-flavored milk and yogurt, frozen sweets such as ice cream and sherbet, powder mixes such as baking mixes and dessert mixes, table sweeteners such as liquid table sweeteners and powdered table sweeteners, dried fish and shellfish products, salted fish and shellfish products, foods boiled in sweetened soy sauce, processed meat and seafood products such as ham, bacon, and sausage, seasonings such as dressings, sauces, soy sauce, miso, sweet sake, Worcester sauce, ketchup, and tsuyu (soup-like sauce) for dipping noodles, spices such as curry powder, processed grain products such as instant noodles, and cereals; pharmaceuticals typified by tablet pharmaceuticals, powder pharmaceuticals, syrup pharmaceuticals, and drop pharmaceuticals; quasi drugs typified by mouth fresheners, gargles, toothpastes, and health drinks; and feeds typified by pet foods, liquid feeds, and powdered feeds. In particular, from the viewpoint of maintaining the quality and stability of the sweetness of monatin, preferred are drinks, foods, pharmaceuticals, quasi drugs, and feeds in which monatin is maintained in the crystalline form, more preferred are powdered drinks, confectionery, powdered mixes, powdered table sweeteners, tablet pharmaceuticals, powder pharmaceuticals, and powdered feeds, and further more preferred are powdered drinks, powdered table sweeteners, and powdered mixes.

The crystal of a multivalent metal salt of monatin or the sweetener composition of the present invention is extremely effective as a preventive or therapeutic agent for metabolic syndromes, a preventive or therapeutic agent for obesity, a preventive or therapeutic agent for diabetes, and an anticaries agent, and it also additionally has a synergistic sweetening effect, a synergistic flavoring effect, a bitterness-masking effect, and a stabilizing effect against photodecomposition.

### EXAMPLES

Hereinafter, the present invention will be described in detail with reference to Examples, however, the present invention is not limited to these Examples.

### [Measurement Method]

First, each measurement method will be described.

### [Method for Measuring Powder X-Ray Diffraction]

1) 0.5 g of sample crystals were collected and ground for 60 seconds in an agate mortar. The obtained powder was placed on a glass plate, and pressure was applied from above to level the powder. The powder was then immediately placed in a powder X-ray diffractometer and the measurement was performed under the following conditions.

2) By using an X-ray diffractometer PW3050 manufactured by Spectris Co., Ltd., the measurement of powder X-ray diffraction with a Cu—Kα radiation was performed under the following conditions: tube: Cu, tube current: 30 mA, tube voltage: 40 kV, sampling width: 0.020°, scan rate: 3°/min, wavelength: 1.54056 Å, and measurement diffraction angle range (20): 4 to 30°.

Measurement program: X′PERT DATA COLLECTION

Analysis program: X′PERT High Score

3) The obtained data were plotted in Excel to create a graph and characteristic acute maximum peaks were read over the range of from 4 to 30°. This method has a diffraction angle error of ±0.2°.

### [Method for Measuring Monatin Content]

The molar ratio of (2R,4R)-monatin to calcium or magnesium was determined as a concentration ratio by HPLC measurement under the following conditions of the monatin content in a solution containing a given concentration of crystals of multivalent metal salts of (2R,4R)-monatin under the following conditions.

### <Used Apparatuses>

Pump: LC-9A manufactured by Shimadzu Corporation

Column oven: CTO-10A manufactured by Shimadzu Corporation

Detector: SPD-10A manufactured by Shimadzu Corporation

Autosampler: SIL-9A manufactured by Shimadzu Corporation

Gradientor: LPG-1000 manufactured by Tokyo Rikakikai Co., Ltd.

<Column>: CAPCELL PAK C18 TYPE MGII 5 μm 4.6 mm×250 mm manufactured by Shiseido Co., Ltd.
<Column temperature> 40° C.
<Detection wavelength> 210 nm
<Mobile phase composition>

Liquid A: 20 mM KH₂PO₄/acetonitrile=100/5

Liquid B: only acetonitrile

<Gradient pattern>

TABLE 1

| Time (min) | Liquid A (%) | Liquid B (%) |
|---|---|---|
| 0 | 100 | 0 |
| 15 | 100 | 0 |
| 40 | 63 | 37 |
| 45 | 63 | 37 |

<Retention time>

(2S,4R)-monatin: 11.8 minutes

(2R,4R)-monatin: 15.1 minutes

<Injection amount> 10 μL
<Analysis cycle> 70 min/sample
<Standard substance for measuring monatin content>

Crystals of (2R,4R)-monatin potassium salt monohydrate having a molecular weight of 348.4

Monatin content in a solution of crystals of multivalent metal salt of (2R,4R)-monatin=(292.3/348.4)×(Wstd×Qs)/(Ws×Qstd)×100(%)

Wstd: Concentration of standard substance (mg/mL)

Qstd: Area value of standard substance

Ws: Concentration of a multivalent metal salt of (2R,4R)-monatin (mg/mL)

Qs: Area value of a multivalent metal salt of (2R,4R)-monatin

(2R,4R)-monatin in the free form having a molecular weight of 292.3

(Note that in the case of a hydrate or a solvate, the mass thereof is also converted.)

### [Method for Measuring Calcium or Magnesium Ions]

The molar ratio of (2R,4R)-monatin to calcium or magnesium was determined under the following conditions as a concentration ratio by measuring the concentration of calcium ions or magnesium ions in a solution containing a given concentration of crystals of multivalent metal salts of (2R,4R)-monatin using an ion chromatograph under the following conditions.

<Name of device> Ion chromatograph IC-2001 manufactured by Toso Co., Ltd.
<Cation measurement column> TSKgel SuperIC-Cation, inner diameter: 4.6 mm, length: 150 mm, manufactured by Toso Co., Ltd.
<Guard column> TSKguard column SuperIC-C, inner diameter: 4.6 mm, length: 10 mm, manufactured by Toso Co., Ltd.
<Eluent> 2.2 mmol/L methanesulfonic acid+1.0 mmol/L 18-crown-6+0.5 mmol/L histidine
<Column temperature>40° C.
<Flow rate> 1 mL/min
<Standard solution> Calcium chloride or magnesium chloride (special grade reagent) was dissolved in pure water and the resulting solution was used as a standard solution.

### [Method for Measuring 1H-NMR Spectra]

<Name of device> AVANCE 400 manufactured by Bruker Co., Ltd. 1H; 400 MHz
<Solvent> Deuterium oxide
<Temperature> Room temperature
<Concentration> About 1% by mass

### [Method for Measuring MS Spectra]

<Name of device> TSQ 700 manufactured by Thermo Quest, Inc.
<Measurement mode> ESI mode

### [Method for Measuring Water Content]

The concentration of water in a solution containing a given concentration of crystals of multivalent metal salts of (2R,4R)-monatin was measured by the Karl Fischer method under the following conditions, and the molar ratio of (2R,4R)-monatin to water was calculated from the amount of the obtained titrant.

<Name of device> Automatic water content measurement apparatus AQV-2000, manufactured by Hiranuma Sangyo Co., Ltd.
<Titrant> Hydranal-composite 5 (manufactured by Riedel-deHaen)<
<Solvent> 150 mL of methanol
<Temperature> Room temperature
<Sample amount> 30 mg

### [Method for Measuring Solvent Content]

A molar ratio of each solvent relative to monatin was calculated on the basis of the ratio of the proton integration value per 1H attributed to the substituent in question of each solvent to the proton integration value per 1H attributed to methylene (δ: 2 ppm, 1H) at the 3′-position of monatin using ¹H-NMR spectra described above.

| | Chemical shift | Attribution |
|---|---|---|
| Solvent | (ppm) | (substituent in question) |
| Methanol | 3.26 | 3H (methyl group) |
| Ethanol | 1.08-1.11 | 3H (methyl group) |
| THF | 1.78-1.81 | 4H (methylene group at the |
| | | 3′- and 4′- positions) |
| i-PrOH | 1.07-1.08 | 6H (methyl group) |
| DMF | 2.77-2.92 | 6H (methyl group) |

### [Method for Measuring Transmittance of Monatin Solution]

<Name of device> UV-visible spectrophotometer, Cary 50, manufactured by Varian Medical Systems, Inc.
<Measurement wavelength>430 nm

### <Temperature>25° C.

<Sample> 1 g of sweetener composition is totally dissolved in 50 mL of water.

### [Water Vapor Adsorption-Desorption Measurement]

<Device> automatic water vapor adsorption analyzer, Belsorp-18, manufactured by BEL Japan, Inc.
<Measurement method> volumetric gas adsorption method
<Adsorption gas> H₂O
<Temperature in air constant temperature bath (K)>353
<Adsorption temperature (K)>298
<Saturation vapor pressure (kPa)>3.169
<Adsorption cross-sectional area (nm²)>0.125
<Maximum adsorption pressure (relative pressure P/P0)> desorption: 0.90, adsorption: 0.95
<Minimum adsorption pressure (relative pressure P/P0)> desorption: 0.10, adsorption: 0.05
<Balancing time>500 sec

### Production Example 1

### Preparation of crystals of (2R,4R)-Monatin Potassium Salt

In accordance with Example 17 described in WO 2003-045914 (Patent document 6), 10 g of crystals of (2R,4R)-monatin monopotassium salt were prepared.

### Production Example 2

### Preparation of crystals of (2R,4R)-monatin in the free form

After 40 g (109 mmol) of crystals of (2R,4R)-monatin potassium salt produced in Production Example 1 was dissolved in 700 mL of water, 54.5 ml of a 1 M aqueous sulfuric acid solution was added dropwise thereto over 2 hours while maintaining the former solution at 10° C. The deposited crystals were separated by filtration, and vacuum drying was performed overnight at 40° C., whereby 31.5 g of (2R,4R)-monatin in the free form was prepared.

### Example 1

### Preparation of Crystals of 5-hydrate of ((2R,4R)-monatin)₂ Calcium Salt pentahydrate

After 5 g (13.7 mmol) of crystals of the (2R,4R)-monatin monopotassium salt produced in Production Example 1 was dissolved in 75 mL of water, 0.758 g (6.83 mmol) of calcium chloride was added thereto at 50° C. To the monatin solution, 75 mL of ethanol was added, and the resulting mixture was stirred at 50° C. for 3 hours. Thereafter, the mixture was cooled to 25° C. over 2.5 hours, and stirring was performed at 25° C. for an additional 10 hours. The deposited crystals were separated by filtration, and vacuum drying was performed at 40° C. The thus obtained dried crystals were stored for 24 hours in a constant temperature and constant humidity device at 44° C. and 78%, whereby 4.6 g of desired calcium salt crystals were obtained.

1HNMR (D2O) δ: 1.94-2.01 (q 1H), 2.57-2.61 (q 1H), 2.99-3.03 (d 1H), 3.19-3.23 (d 1H), 3.54-3.57 (q 1H), 7.05-7.17 (m 3H), 7.40-7.42 (m 1H), 7.64-7.66 (m 1H)

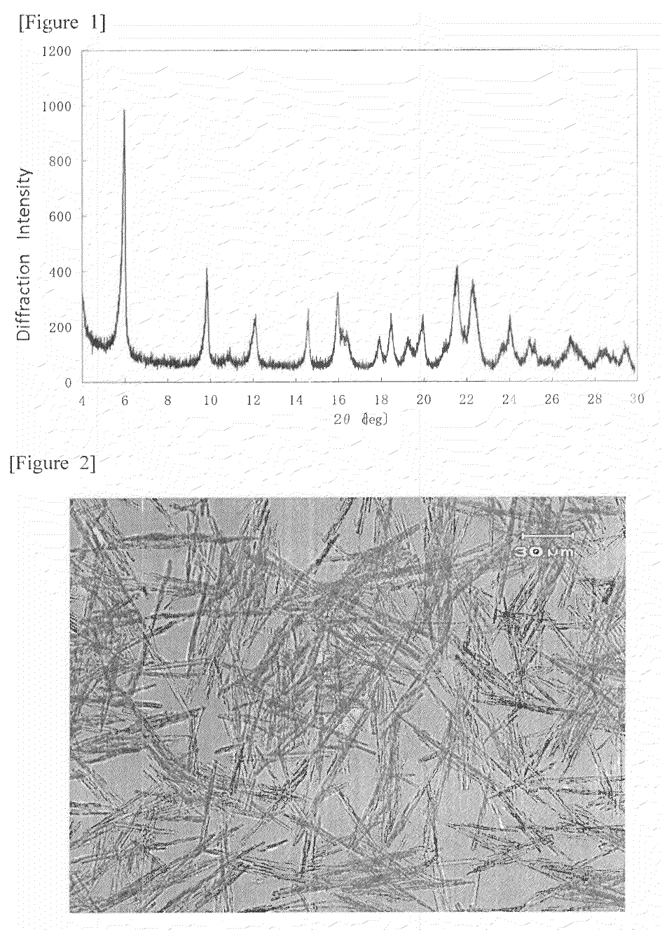

ESI-MS: 293.1 (M+H)⁺, 291.1 (M−H)⁻

<Moisture content> 13.5 wt % (corresponding to the following equation: monatin:water=2:5)<
<Calcium content> 5.6 wt % (corresponding to the following equation: monatin:calcium=2:1)<
<Characteristic X-ray diffraction peaks (2θ±0.2°, CuKα)>6.0°, 9.8°, 16.0°, 21.5° and 22.3° (FIG. 1)<
<Crystalline form> The crystalline form in the deposited ML was a relatively small needle-like form (FIG. 2)

### Example 2

### Preparation of Crystals of ((2R,4R)-monatin)₂ Magnesium Salt tetrahydrate

After 10 g (28.5 mmol) of crystals of the (2R,4R)-monatin monopotassium salt produced in Production Example 1 was dissolved in 20 mL of water, 28.3 mL of an aqueous solution of 501 mM magnesium chloride was added thereto at room temperature. After the monatin solution was stirred at 25° C. for 18 hours, 80 g of methanol was added thereto, and the resulting mixture was stirred at room temperature for 6 hours. The deposited crystals were separated by filtration, and the obtained slurry was washed for 2 hours with 50 g of 80% methanol, followed by filtration. Thereafter, vacuum drying was performed at 40° C. The thus obtained dried crystals were stored for 24 hours in a constant temperature and constant humidity device at 44° C. and 78%, followed by vacuum drying at 40° C., whereby 8.8 g of desired magnesium salt crystals were obtained.

1HNMR (D2O) δ: 1.94-2.01 (q 1H), 2.57-2.61 (q 1H), 2.99-3.03 (d 1H), 3.19-3.23 (d 1H), 3.54-3.57 (q 1H), 7.05-7.17 (m 3H), 7.40-7.42 (m 1H), 7.64-7.66 (m 1H)

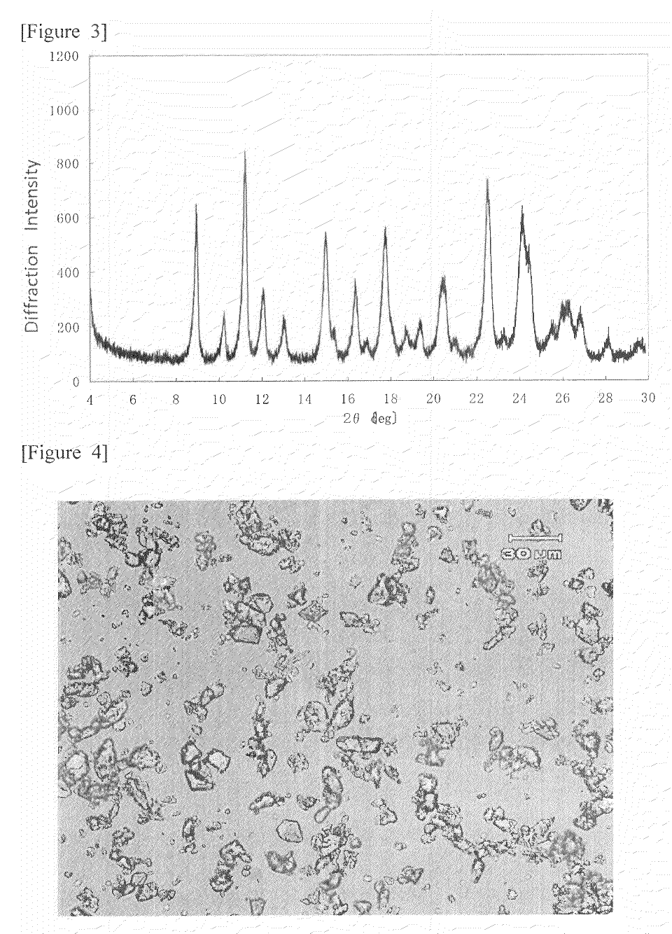

### ESI-MS: 293.1 (M+H)⁺, 291.1 (M−H)⁻

<Moisture content> 10.8 wt % (corresponding to the following equation: monatin:water=2:4)
<Magnesium content> 3.6 wt % (corresponding to the following equation: monatin:magnesium=2:1)<
<Characteristic X-ray diffraction peaks (2θ±0.2°, CuKα)> 8.9°, 11.2°, 15.0°, 17.8°, and 22.5° (FIG. 3)<
<Crystalline form> The crystalline form in the deposited ML was a relatively small flake-like form (FIG. 4)<
<Sweetness intensity> 2700 times higher (in comparison with the sweetness intensity of an aqueous solution of 5% sucrose, average of the sweetness intensities evaluated by 7 panelists)

### Example 3

### Preparation of Crystals of 4.6-hydrate 0.67-ethanol solvate of ((2R,4R)-monatin)₂ Calcium Salt

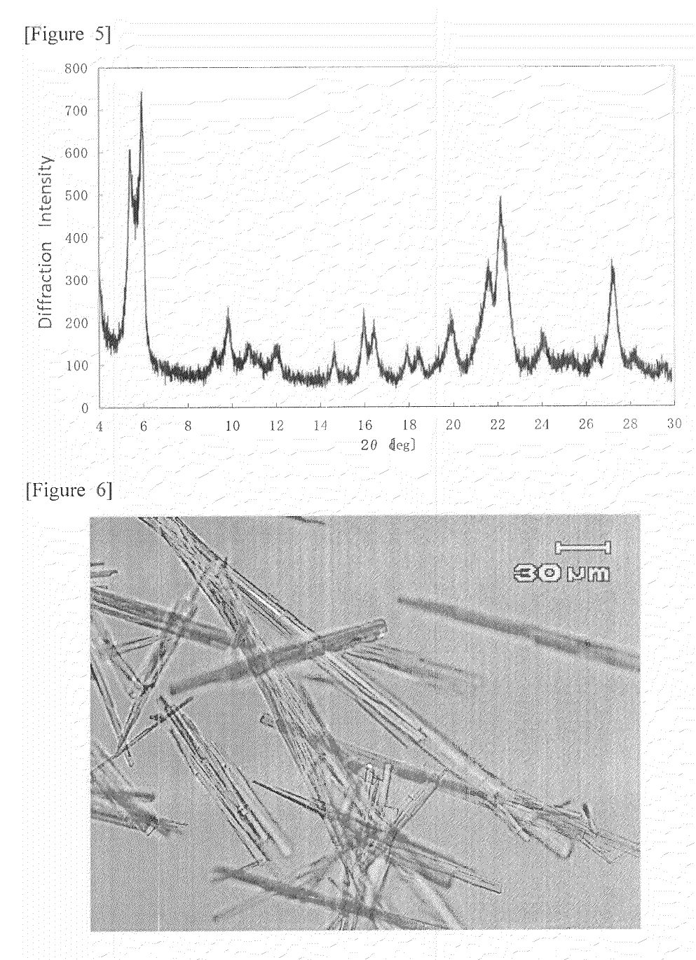

After 15 g (41 mmol) of crystals of (2R,4R)-monatin monopotassium salt was dissolved in 225 mL of water, 2.274 g (20.5 mmol) of calcium chloride was added thereto. After the monatin solution was heated to 50° C., 75 mL of ethanol was added thereto, and the resulting mixture was stirred for 1.5 hours. Thereafter, the mixture was cooled to 25° C. over 2.5 hours, and stirring was performed at 25° C. for an additional 12.5 hours. The deposited crystals were separated by filtration, and vacuum drying was performed at 40° C., whereby 14.1 g of desired calcium salt crystals were obtained.

<Moisture content> 12.2 wt % (corresponding to the following equation: monatin:water=1.9:4.6)<
<Calcium content> 5.8 wt % (corresponding to the following equation: monatin:calcium=1.9:1)<
<EtOH content> 4.5 wt % (corresponding to the following equation: monatin:EtOH=1.9:0.67)<
<Characteristic X-ray diffraction peaks (2θ±0.2°, CuKα)> 5.4°, 6.0°, 16.4°, 22.2°, and 27.3° (FIG. 5)<
<Crystalline form> The crystalline form in the deposited ML was a needle-like form (FIG. 6).

### Example 4

### Preparation of Crystals of 5.7-hydrate of ((2R,4R)-monatin)₂ Calcium Salt

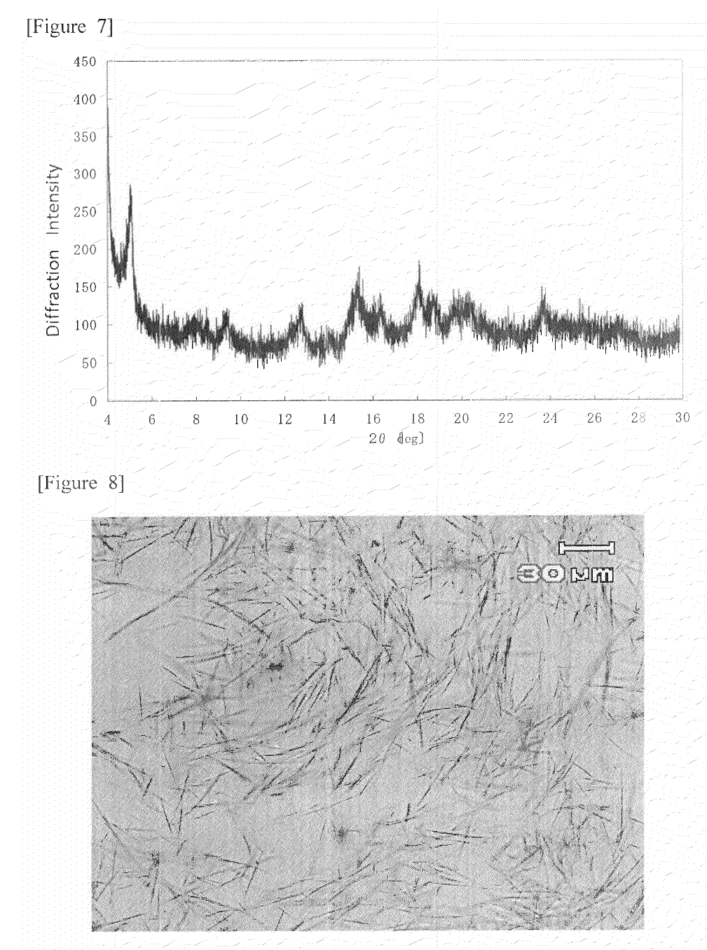

After 0.4 g (1.08 mmol) of crystals of 5-hydrate of (2R,4R)-monatin)₂ calcium salt was dissolved in 8.5 mL of water, the resulting solution was heated to 65° C. and 8.5 mL of CH₃CN was added thereto, and the resulting mixture was stirred at 45° C. for 12 hours. The deposited crystals were separated by filtration, and vacuum drying was performed at 40° C., whereby 0.288 g of calcium salt crystals were obtained.

<Moisture content> 12.6 wt % (corresponding to the following equation: monatin:water=2.3:5.7)<
<Calcium content> 4.9 wt % (corresponding to the following equation: monatin:calcium=2.3:1)<
<CH₃CN content> 0 wt %<
<Characteristic X-ray diffraction peaks (2θ±0.2°, CuKα)> 5.0°, 12.8°, 15.3°, 18.1°, and 23.7° (FIG. 7)<
<Crystalline form> The crystalline form in the deposited ML was a small needle-like form (FIG. 8).

### Example 5

### Preparation of Crystals of 5.9-hydrate 0.72-THF solvate of ((2R,4R)-monatin)₂ Calcium Salt

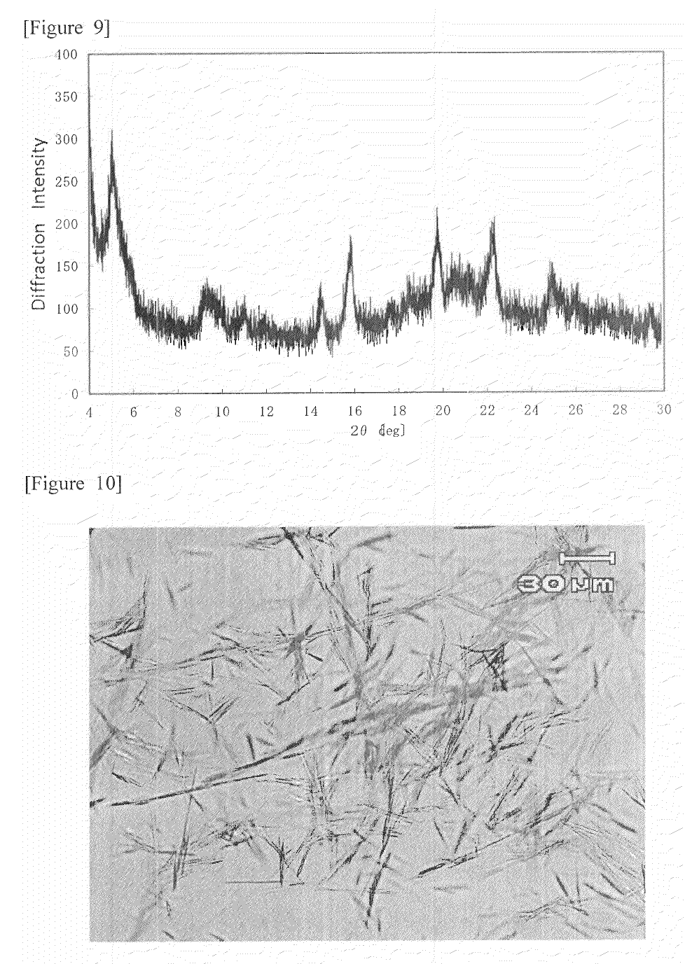

After 0.4 g (1.08 mmol) of crystals of 5-hydrate of (2R,4R)-monatin)₂ calcium salt was dissolved in 8.5 mL of water, the resulting solution was heated to 65° C. and 8.5 mL of THF was added thereto, and the resulting mixture was stirred at 45° C. for 12 hours. The deposited crystals were separated by filtration, and vacuum drying was performed at 40° C., whereby 0.288 g of calcium salt crystals were obtained.

<Moisture content> 11.4 wt % (corresponding to the following equation: monatin:water=2.5:5.9)<
<Calcium content> 4.3 wt % (corresponding to the following equation: monatin:calcium=2.5:1)<
<THF content> 5.6 wt % (corresponding to the following equation: monatin:THF=2.5:0.72)<
<Characteristic X-ray diffraction peaks (2θ±0.2°, CuKα)> 5.1°, 15.9°, 19.7°, and 22.3° (FIG. 9)<
<Crystalline form> The crystalline form in the deposited ML was a small needle-like form (FIG. 10).

### Example 6

### Preparation of Crystals of 3.8-hydrate 0.63-i-PrOH solvate of ((2R,4R)-monatin)₂ Calcium Salt

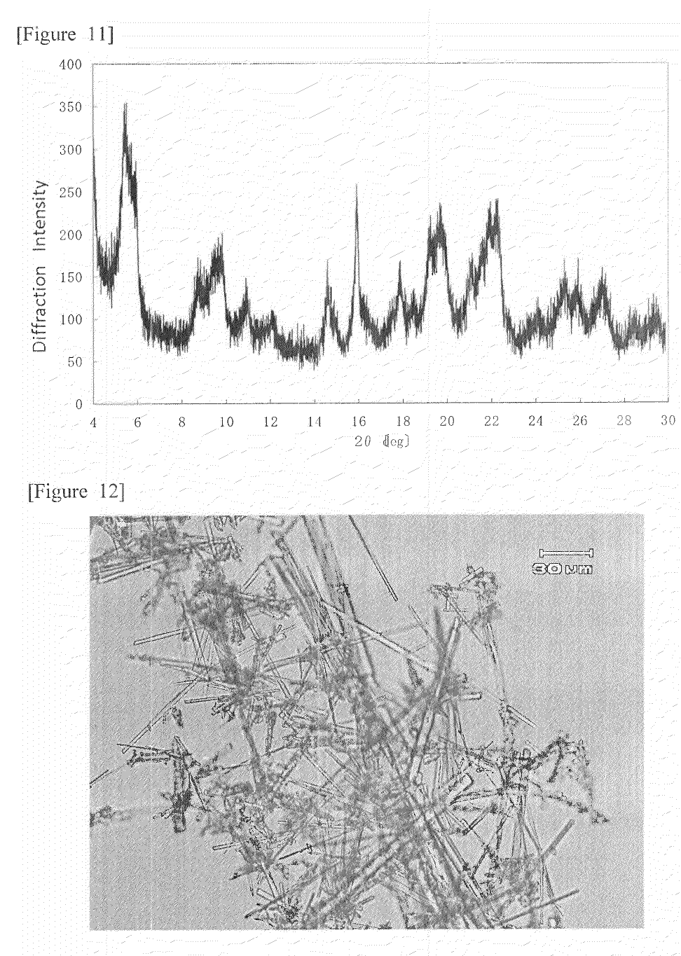

After 0.4 g (1.08 mmol) of crystals of 5-hydrate of ((2R,4R)-monatin)₂ calcium salt was dissolved in 8.5 mL of water, the resulting solution was heated to 65° C. and 8.5 mL of i-PrOH was added thereto, and the resulting mixture was stirred at 45° C. for 25 hours. The deposited crystals were separated by filtration, and vacuum drying was performed at 40° C., whereby 0.337 g of calcium salt crystals were obtained.

<Moisture content> 10.67 wt % (corresponding to the following equation: monatin:water=1.7:3.8)<
<Calcium content> 6.2 wt % (corresponding to the following equation: monatin:calcium=1.7:1)<
<i-PrOH content> 5.87 wt % (corresponding to the following equation: monatin:i-PrOH=1.7:0.63)<
<Characteristic X-ray diffraction peaks (2θ±0.2°, CuKα)>5.4°, 15.9°, 19.7°, and 22.3° (FIG. 11)<
<Crystalline form> The crystalline form in the deposited ML was a needle-like form (FIG. 12).

### Example 7

### Preparation of Crystals of 7.5-hydrate of ((2R,4R)-monatin)₂ Magnesium Salt

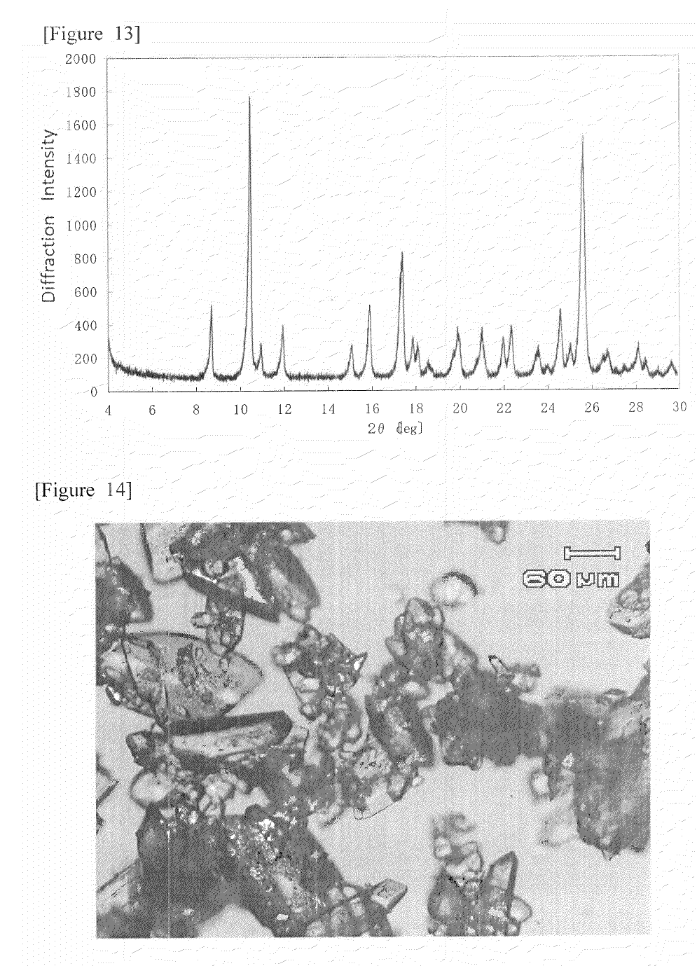

After 30 g (100 mmol) of crystals of (2R,4R)-monatin in the free form was dispersed in 300 mL of water, 3.36 g (58 mmol) of magnesium hydroxide was added thereto at 40° C. The resulting mixture was stirred at 40° C. for 4 hours, and thereafter stirred at 25° C. for 16 hours. The deposited crystals (38.38 g) were separated by filtration, and vacuum drying was performed at 40° C., whereby 28.9 g of magnesium salt crystals were obtained.

<Moisture content> 18.34 wt % (corresponding to the following equation: monatin:water=2:7.5)<
<Magnesium content> 3.67 wt % (corresponding to the following equation: monatin:magnesium=2:1)<
<Characteristic X-ray diffraction peaks (2θ±0.2°, CuKα)>8.7°, 10.5°, 15.9°, 17.4°, 21.0°, and 25.6° (FIG. 13)<Crystalline form> The crystalline form in the deposited ML was a columnar form (FIG. 14).

### Example 8

### Preparation of Crystals of ((2R,4R)-monatin)₂ Magnesium Salt Dihydrate

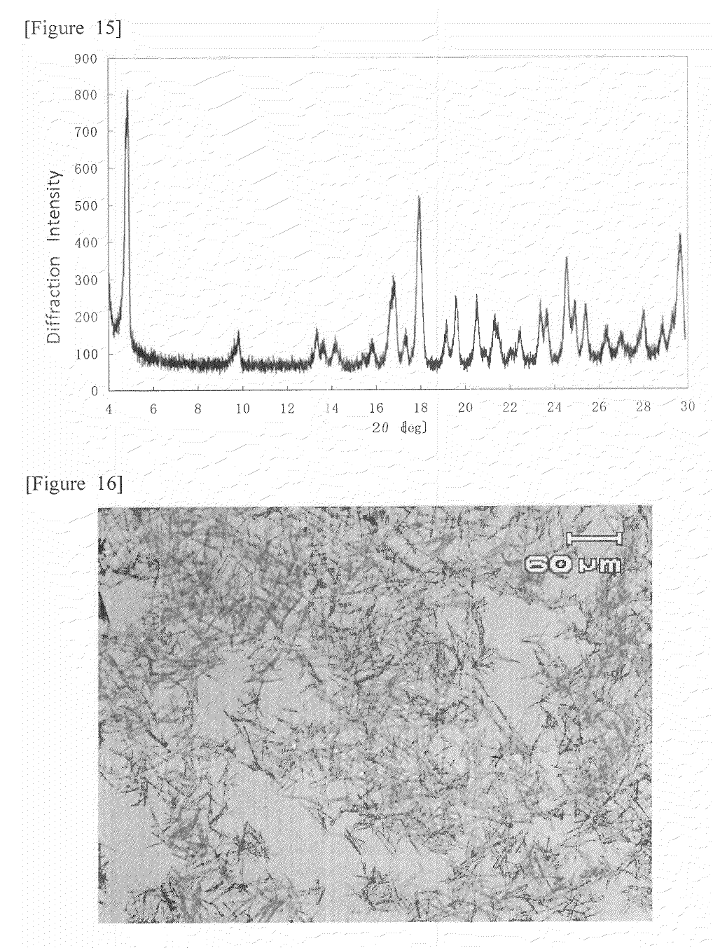

After 120 g (345 mmol) of crystals of (2R,4R)-monatin potassium salt was dissolved in 150 mL of water, 4.15 g (34.5 mmol) of magnesium sulfate was added thereto at 60° C. Further, an aqueous solution (100 mL of water) containing 16.61 g (138 mmol) of magnesium sulfate was added thereto over 6.4 hours. After completion of addition, the deposited crystals were separated by filtration and washed with 100 mL of water, whereby wet crystals (204.7 g) were obtained. The obtained wet crystals were subjected to vacuum drying at 40° C., whereby 105 g of magnesium salt crystals were obtained. Further, in order to remove potassium sulfate which was contained therein in a small amount, 400 mL of water was added to 105 g of the dried crystals, and the resulting mixture was stirred at 25° C. for 1.5 hours. The thus obtained slurry was separated by filtration and washed with 300 mL of water, whereby wet crystals (153.9 g) were obtained. The obtained wet crystals were subjected to vacuum drying at 40° C., whereby 85.7 g of magnesium salt crystals were obtained.

<Characteristic X-ray diffraction peaks (2θ±0.2°, CuKα)> 4.9°, 16.8°, 18.0°, and 24.6° (FIG. 15)<
<Moisture content> 6.0 wt % (corresponding to the following equation: monatin:water=1:1)<
<Magnesium content> 3.61 wt % (corresponding to the following equation: monatin:magnesium=2:1)<
<Crystalline form> The crystalline form in the deposited ML was a microcrystalline form (FIG. 16).

### Example 9

### Preparation of Crystals of 3.1-hydrate 2.4-ethanol Solvate of ((2R,4R)-monatin)₂ Magnesium Salt

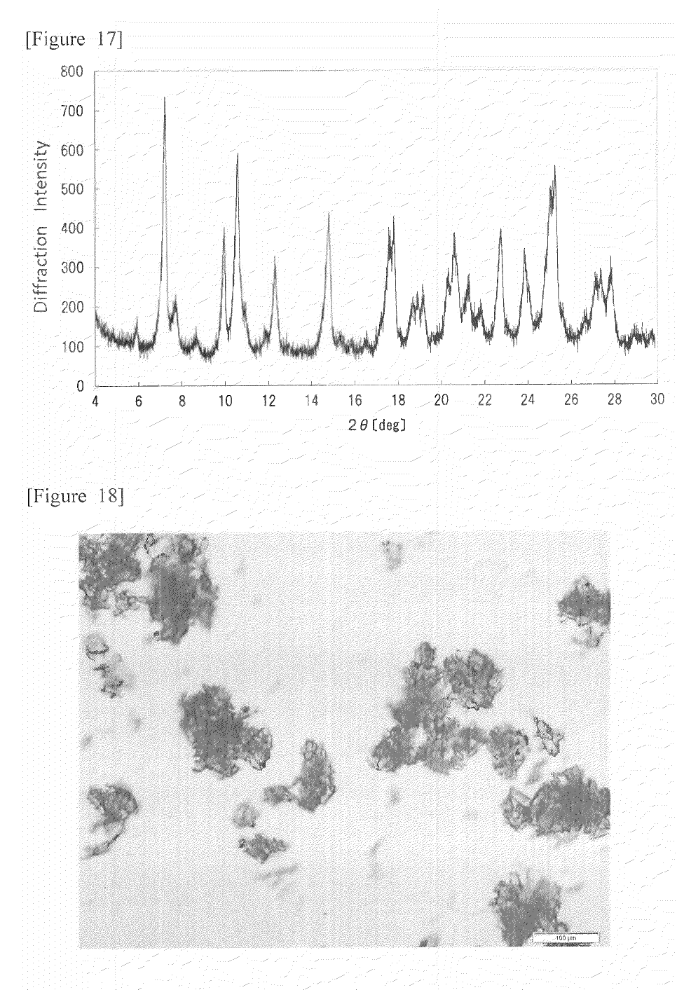

After 10 g (33.3 mmol) of crystals of (2R,4R)-monatin in the free form was dispersed in 100 mL of water, 0.971 g (16.7 mmol) of magnesium hydroxide was added thereto at 25° C., and the resulting mixture was stirred for 3 hours. Then, 506 mL of ethanol was further added dropwise thereto over about 3 hours, and the resulting mixture was stirred at 25° C. for 25.5 hours. The deposited crystals (17.73 g) were separated by filtration, and vacuum drying was performed at room temperature, whereby 12.04 g of desired magnesium salt crystals were obtained.

<Moisture content> 6.7 wt % (corresponding to the following equation: monatin:water=2.1:3.1)<
<Magnesium content> 2.9 wt % (corresponding to the following equation: monatin:magnesium=2.1:1)<
<EtOH content> 13.5 wt % (corresponding to the following equation: monatin:EtOH=2.1:2.4)<
<Characteristic X-ray diffraction peaks (2θ±0.2°, CuKα)>7.2°, 10.0°, 10.6°, 12.3°, 14.8°, 17.8°, and 25.3° (FIG. 17)<

<Crystalline form> The crystalline form in the deposited ML was a microcrystalline form (FIG. 18).

### Example 10

### Preparation of Crystals of 7.2-hydrate 0.23-methanol Solvate of ((2R,4R)-monatin)₂ Magnesium Salt

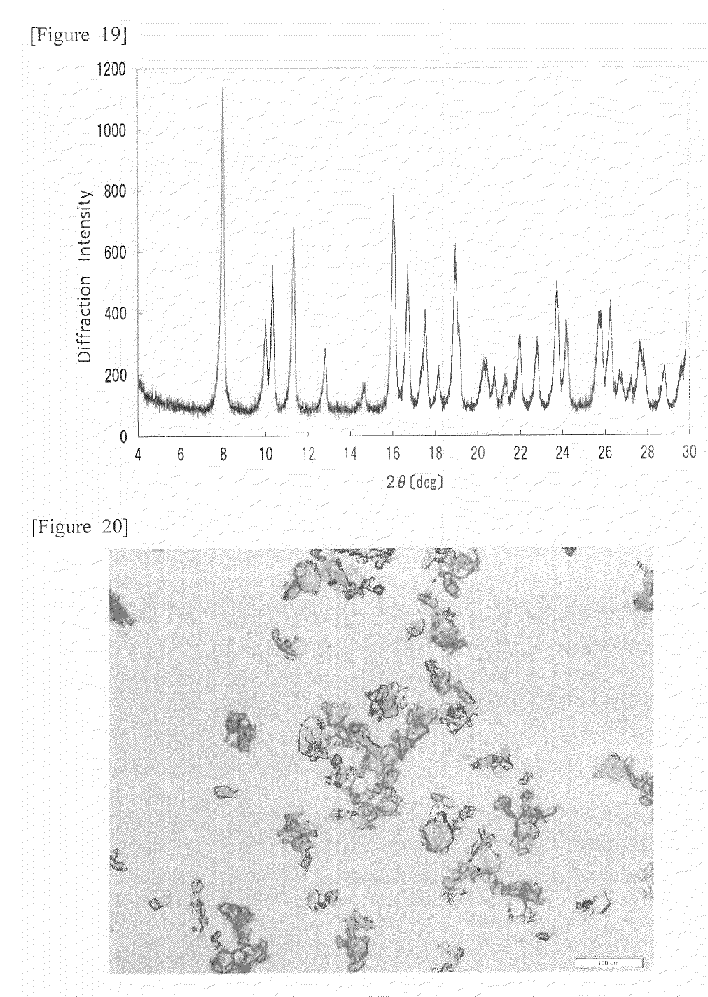

After 10 g (33.3 mmol) of crystals of (2R,4R)-monatin in the free form was dispersed in 100 mL of water, 0.971 g (16.7 mmol) of magnesium hydroxide was added thereto at 25° C., and the resulting mixture was stirred for 3 hours. Then, 506 mL of methanol was further added dropwise thereto over about 3 hours, and the resulting mixture was stirred at 25° C. for 20 hours. The deposited crystals (15.87 g) were separated by filtration, and vacuum drying was performed at room temperature, whereby 11.94 g of desired magnesium salt crystals were obtained.

<Moisture content> 18.16 wt % (corresponding to the following equation: monatin:water=1.9:7.2)<
<Magnesium content> 3.3 wt % (corresponding to the following equation: monatin:magnesium=1.9:1)<
<Methanol content> 1.0 wt % (corresponding to the following equation: monatin:methanol=1.9:0.23)<
<Characteristic X-ray diffraction peaks (2θ±0.2°, CuKα)> 8.0°, 10.0°, 10.3°, 11.4°, 16.1°, 19.0°, and 23.7° (FIG. 19)<
<Crystalline form> The crystalline form in the deposited ML was a microcrystalline form (FIG. 20).

### Example 11

### Preparation of Crystals of 8.5-hydrate 2.5-DMF Solvate of ((2R,4R)-monatin)₂ Magnesium Salt

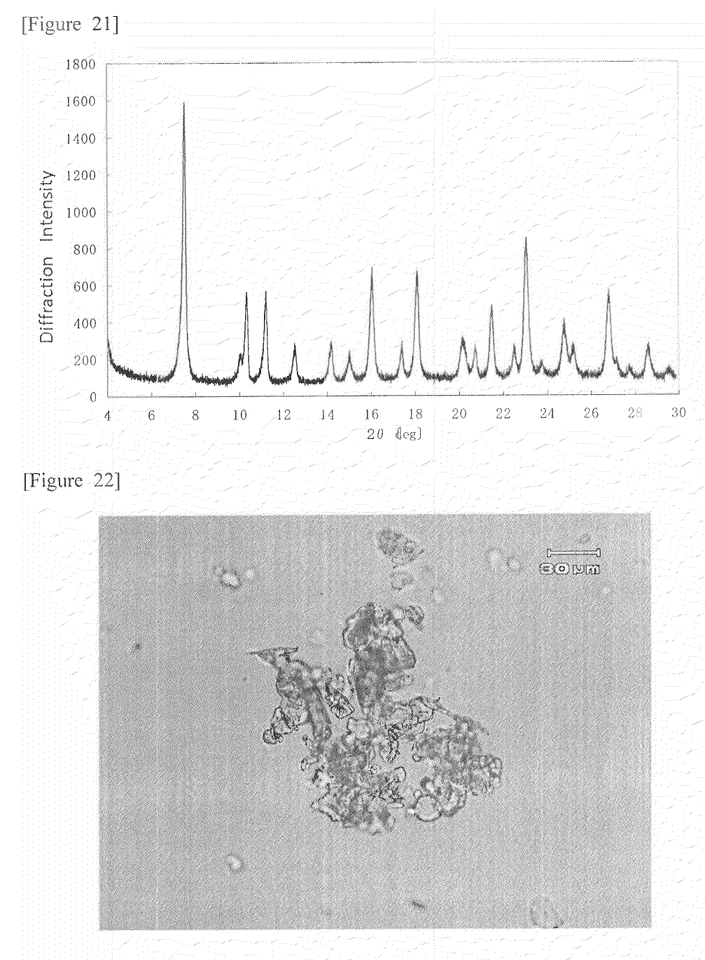

After 0.4 g (0.56 mmol) of crystals of ((2R,4R)-monatin)₂ magnesium salt tetrahydrate was dissolved in 10 mL of water, 10 mL of DMF was added thereto, and the resulting mixture was stirred at 45° C. for 47 hours. The deposited crystals were separated by filtration, and vacuum drying was performed at 40° C., whereby 0.212 g of magnesium salt crystals were obtained.

<Moisture content> 7.46 wt % (corresponding to the following equation: monatin:water=2.5:8.5)<
<Magnesium content> 2.9 wt % (corresponding to the following equation: monatin:magnesium=2.5:1)<
<DMF content> 2.7 wt % (corresponding to the following equation: monatin:DMF=2.5:2.5)<
<Characteristic X-ray diffraction peaks (2θ±0.2°, CuKα)> 7.5°, 10.3°, 11.2°, 16.0°, 18.1°, and 23.0° (FIG. 21)<
<Crystalline form> The crystalline form in the deposited ML was a microcrystalline form (FIG. 22).

### Example 12

### Preparation of Crystals of ((2R,4R)-monatin)₂ Magnesium Salt Nonahydrate

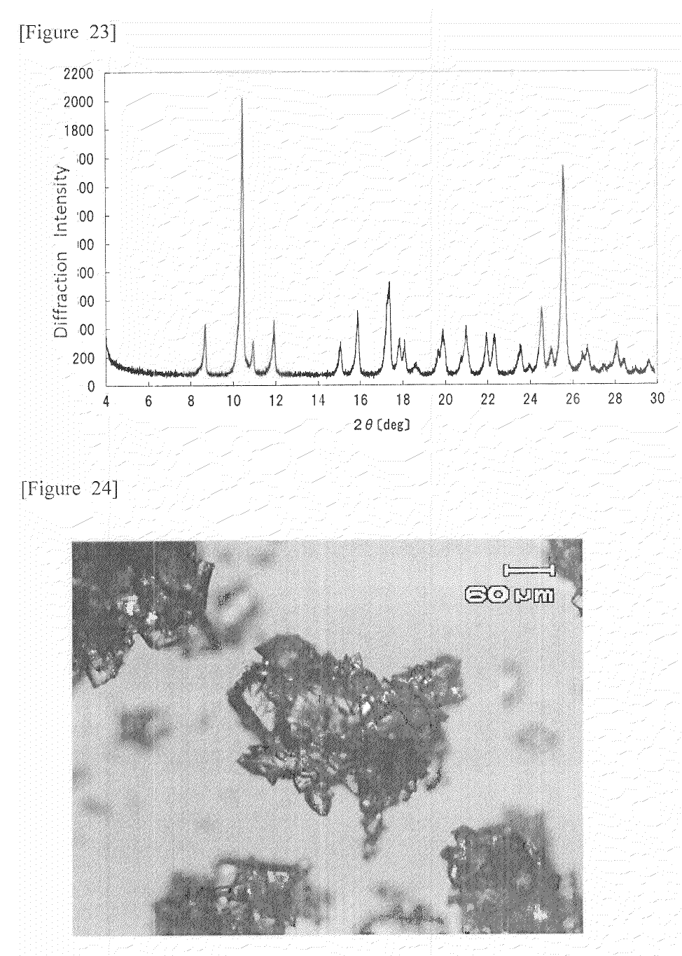

After 30 g (100 mmol) of crystals of (2R,4R)-monatin in the free form was dispersed in 300 mL of water, 3.21 g (55 mmol) of magnesium hydroxide was added thereto at 65° C., and the resulting mixture was stirred at 65° C. for 1 hour. The deposited crystals (27.28 g) were separated by filtration, and vacuum drying was performed at 40° C. for 4 hours, whereby 22.29 g of magnesium salt crystals were obtained.

<Moisture content> 21.22 wt % (corresponding to the following equation: monatin:water=2:9)<
<Magnesium content> 3.45 wt % (corresponding to the following equation: monatin:magnesium=2:1)<
<Characteristic X-ray diffraction peaks (2θ±0.2°, CuKα)> 8.7°, 10.5°, 15.9°, 17.4°, 21.0°, and 25.6° (FIG. 23)<
<Crystalline form> The crystalline form in the deposited ML was a columnar form (FIG. 24).

### Example 13

### Water Vapor Adsorption-Desorption Curve for Crystals

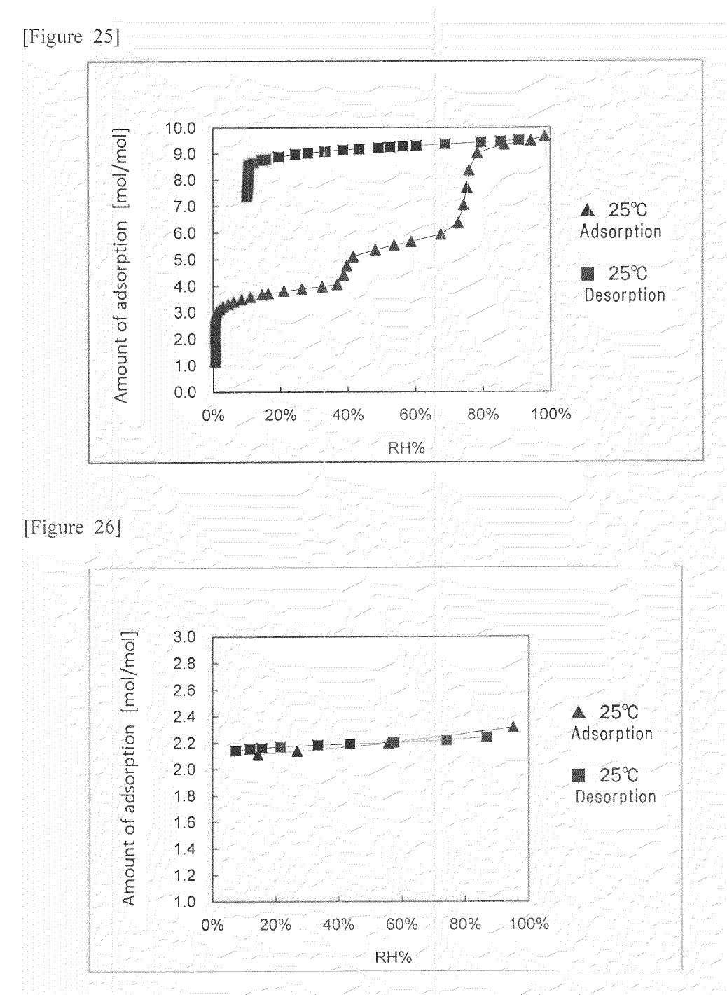

The water vapor adsorption-desorption curves for the crystals of ((2R,4R)-monatin)₂ magnesium salt tetrahydrate obtained by the method described in Example 2 and the crystals of ((2R,4R)-monatin)₂ magnesium salt dihydrate obtained by the method described in Example 8 were determined. The measurement values are shown in FIGS. 25 and 26.

### Test Example 1

### Evaluation of Storage Stability (when Blending Glucose)

The crystals of ((2R,4R)-monatin)₂ calcium salt pentahydrate obtained in Example 1, the crystals of ((2R,4R)-monatin)₂ magnesium salt tetrahydrate obtained in Example 2, the crystals of 4.6-hydrate 0.67-ethanol solvate of ((2R,4R)-monatin)₂ calcium salt obtained in Example 3, the crystals of ((2R,4R)-monatin)₂ magnesium salt dihydrate obtained in Example 8, the crystals of monoethanol solvate of ((2R,4R)-monatin)₂ magnesium salt obtained in Example 9, the crystals of 0.23-methanol solvate of ((2R,4R)-monatin)₂ magnesium salt obtained in Example 10, and the crystals of monatin monopotassium salt obtained in Production Example 1 were evaluated with respect to storage stability by the following method.

TABLE 2
Compositional Table 1 (glucose)

| | | | | | | | Comparative |
|---|---|---|---|---|---|---|---|
| | Compo- | Compo- | Compo- | Compo- | Compo- | Compo- | Compo- |
| | sitional | sitional | sitional | sitional | sitional | sitional | sitional |
| Ingredient (g) | Example 1 | Example 2 | Example 3 | Example 4 | Example 5 | Example 6 | Example 1 |
| [Example 1] Crystals of | 0.17 | | | | | | |
| ((2R,4R)-monatin)₂ calcium salt | | | | | | | |
| pentahydrate | | | | | | | |
| [Example 2] Crystals of | | 0.16 | | | | | |
| ((2R,4R)-monatin)₂ magnesium salt | | | | | | | |
| tetrahydrate | | | | | | | |
| [Example 3] Crystals of 4.6-hydrate | | | 0.17 | | | | |
| 0.67-ethanol solvate of | | | | | | | |
| ((2R,4R)-monatin)₂ calcium salt | | | | | | | |
| [Example 8] Crystals of | | | | 0.15 | | | |
| ((2R,4R)-monatin)₂ magnesium salt | | | | | | | |
| dihydrate | | | | | | | |
| [Example 9] Crystals of | | | | | 0.19 | | |
| monoethanol solvate of | | | | | | | |
| ((2R,4R)-monatin)₂ magnesium salt | | | | | | | |
| [Example 10] Crystals of | | | | | | 0.19 | |
| 0.23-methanol solvate of | | | | | | | |
| ((2R,4R)-monatin)₂ magnesium salt | | | | | | | |
| [Production Example 1] Crystals of | | | | | | | 0.17 |
| (2R,4R)-monatin monopotassium | | | | | | | |
| salt | | | | | | | |
| Maltodextrin | 0.89 | 0.90 | 0.89 | 0.91 | 0.89 | 0.88 | 0.89 |
| Glucose | 28.94 | 28.94 | 28.94 | 28.95 | 28.94 | 28.94 | 28.94 |
| Total weight (g) | 30.00 | 30.00 | 30.00 | 30.00 | 30.00 | 30.00 | 30.00 |

### [Storage Conditions]

1 g of each sweetener composition shown in the compositional table 1 was filled in a paper packaging material, and the packaging material was sealed with a heat seal. The composition was stored for a given period of time in a constant temperature and constant humidity device at 44° C. and 78%. Then, the total amount of the stored sample was dissolved in 50 mL of water, and the residual ratio of each monatin salt was calculated from the data obtained by HPLC analysis of the solution.

### [Change in Residual Ratio Over Time]

The results of the residual ratio of monatin are shown in Table 3. It was found that the residual ratio under high temperature and high humidity conditions is higher in the case of the calcium salt and the magnesium salt than in the case of the potassium salt. The results of the transmittance of the solution of each product after storage are shown in Table 4. In the case of the potassium salt, the transmittance was decreased and the crystals were colored yellow. However, in the case of the calcium salt and the magnesium salt, a change in color was not observed.

TABLE 3
Residual Ratio of Monatin (products containing glucose)

| | Residual ratio (%) | | | |
|---|---|---|---|---|
| Storage time (week) | 0 | 2 | 4 | 8 |
| [Compositional Example 1] Crystals of | 100 | 95 | 92 | 77 |
| ((2R,4R)-monatin)₂ calcium salt pentahydrate | | | | |
| [Compositional Example 2] Crystals of | 100 | 95 | 91 | 86 |
| ((2R,4R)-monatin)₂ magnesium salt | | | | |
| tetrahydrate | | | | |
| [Compositional Example 3] Crystals of | 100 | 90 | 82 | |
| 4.6-hydrate 0.67-ethanol solvate of ((2R,4R)- | | | | |
| monatin)₂ calcium salt | | | | |
| [Compositional Example 4] Crystals of | 100 | 100 | 100 | 96 |
| ((2R,4R)-monatin)₂ magnesium salt dihydrate | | | | |
| [Compositional Example 5] Crystals of | 100 | 92 | 89 | 83 |
| monoethanol solvate of ((2R,4R)-monatin)₂ | | | | |
| magnesium salt | | | | |
| [Compositional Example 6] Crystals of | 100 | 94 | 88 | 82 |
| 0.23-methanol solvate of ((2R,4R)-monatin)₂ | | | | |
| magnesium salt | | | | |
| [Comparative Compositional Example 1] | 100 | 79 | 56 | 27 |
| Crystals of (2R,4R)-monatin monopotassium | | | | |
| salt | | | | |

TABLE 4
Transmittance of Monatin Solution (products containing glucose)

| | % T (430 nm) | |
|---|---|---|
| Storage time (week) | 2 | 8 |
| [Compositional Example 1] Crystals of | 99 | 98 |
| ((2R,4R)-monatin)2 calcium salt pentahydrate | | |
| [Compositional Example 2] Crystals of | 99 | 99 |
| ((2R,4R)-monatin)2 magnesium salt tetrahydrate | | |
| [Compositional Example 3] Crystals of 4.6-hydrate | 99 | |
| 0.67-ethanol solvate of ((2R,4R)-monatin)2 calcium salt | | |
| [Compositional Example 4] Crystals of | 98 | 98 |
| ((2R,4R)-monatin)2 magnesium salt dihydrate | | |
| [Compositional Example 5] Crystals of | 99 | 98 |
| monoethanol solvate of ((2R,4R)-monatin)2 magnesium | | |
| salt | | |
| [Compositional Example 6] Crystals of | 99 | 96 |
| 0.23-methanol solvate of ((2R,4R)-monatin)2 magnesium | | |
| salt | | |
| [Comparative Compositional Example 1] Crystals of | 98 | 94 |
| (2R,4R)-monatin monopotassium salt | | |

### Test Example 2

### Evaluation of Storage Stability (when Blending Sucrose)

The crystals of ((2R,4R)-monatin)₂ calcium salt pentahydrate obtained in Example 1, the crystals of ((2R,4R)-monatin)₂ magnesium salt tetrahydrate obtained in Example 2, and the crystals of monatin monopotassium salt obtained in Production Example 1 were evaluated with respect to storage stability by the following method.

TABLE 5
Compositional Table 2 (sucrose)

| | | | Comparative |
|---|---|---|---|
| | Compositional | Compositional | Compositional |
| Ingredient (g) | Example 6 | Example 7 | Example 2 |
| [Example 1] Crystals | 0.29 | | |
| of ((2R,4R)-monatin)₂ | | | |
| calcium salt | | | |
| pentahydrate | | | |
| [Example 2] Crystals | | 0.27 | |
| of ((2R,4R)-monatin)₂ | | | |
| magnesium salt | | | |
| tetrahydrate | | | |
| [Production | | | 0.28 |
| Example 1] | | | |
| Crystals of (2R,4R)- | | | |
| monatin | | | |
| monopotassium salt | | | |
| Sucrose | 49.72 | 49.73 | 49.72 |
| Total weight (g) | 50.00 | 50.00 | 50.00 |

### [Storage Conditions]

1 g of each sweetener composition shown in the compositional table 2 was filled in a paper packaging material, and the packaging material was sealed with a heat seal. The composition was stored for a given period of time in a constant temperature and constant humidity device at 44° C. and 78%. Then, the total amount of the stored sample was dissolved in 50 mL of water, and the residual ratio of each monatin salt was calculated from the data obtained by HPLC analysis of the solution.

### [Change in Residual Ratio Over Time]

The results of the residual ratio of monatin are shown in Table 6. It was found that the residual ratio under high temperature and high humidity conditions is higher in the case of the calcium salt and the magnesium salt than in the case of the potassium salt. The results of the transmittance of the solution of each product after storage are shown in Table 7. In the case of the potassium salt, the transmittance was decreased and the crystals were colored yellow. However, in the case of the calcium salt and the magnesium salt, a change in color was not observed.

TABLE 6
Residual Ratio of Monatin (products containing sucrose)

| | Residual ratio (%) | | | | |
|---|---|---|---|---|---|
| Storage time (week) | 0 | 4 | 8 | 13 | 26 |
| [Compositional Example 6] Crystals of | 100 | 98 | 98 | 97 | 93 |
| ((2R,4R)-monatin)2 calcium salt pentahydrate | | | | | |
| [Compositional Example 7] Crystals of | 100 | 99 | 99 | 97 | 95 |
| ((2R,4R)-monatin)2 magnesium salt | | | | | |
| tetrahydrate | | | | | |
| [Comparative Compositional Example 2] | 100 | 91 | 84 | 72 | 44 |
| Crystals of (2R,4R)-monatin monopotassium | | | | | |
| salt | | | | | |

TABLE 7
Transmittance of Monatin Solution (products containing sucrose)

| | % T (430 nm) | | | | |
|---|---|---|---|---|---|
| Storage time (week) | 0 | 4 | 8 | 13 | 26 |
| [Compositional Example 6] Crystals of | 99 | 99 | 98 | 98 | 98 |
| ((2R,4R)-monatin)2 calcium salt pentahydrate | | | | | |
| [Compositional Example 7] Crystals of | 99 | 98 | 98 | 98 | 99 |
| ((2R,4R)-monatin)2 magnesium salt tetrahydrate | | | | | |
| [Comparative Compositional Example 2] Crystals | 98 | 99 | 98 | 98 | 95 |
| of (2R,4R)-monatin monopotassium salt | | | | | |

### Formulation Example 1

### Powdered Table Sweetener

A formulation example of a powdered table sweetener will be shown below.

TABLE 3
(mass %)

| | |
|---|---|
| Crystals of calcium salt described in Example 1 | 0.12 |
| Erythritol | 20 |
| Reduced maltose | Balance |
| Total | 100 |

### Formulation Example 2

### Powdered Mix

A formulation example of a powdered mix will be shown below.

TABLE 4
(mass %)

| | |
|---|---|
| Crystals of magnesium salt described in Example 2 | 0.12 |
| Maltitol | 60 |
| Resistant dextrin | Balance |
| Total | 100 |

### INDUSTRIAL APPLICABILITY

A crystal of a multivalent metal salt of (2R,4R)-monatin has made it possible to provide a stable monatin crystal. The utility and physical properties of stereoisomers thereof as sweeteners were elucidated. Also, the crystal has made it possible to provide oral products such as drinks, foods, pharmaceuticals, quasi drugs, and feeds, each containing a versatile stable and safe crystal of a multivalent metal salt of monatin, which is very significant.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A crystal of a multivalent metal salt of (2R,4R)-monatin.

2. A crystal of a multivalent metal salt of (2R,4R)-monatin according to claim 1, wherein said multivalent metal salt is a divalent metal salt.

3. A crystal of a multivalent metal salt of (2R,4R)-monatin according to claim 2, wherein said multivalent metal salt is an alkaline earth metal salt.

4. A crystal of a multivalent metal salt of (2R,4R)-monatin according to claim 3, wherein said multivalent metal salt is at least one salt selected from a calcium salt and a magnesium salt.

5. A crystal of a multivalent metal salt of (2R,4R)-monatin according to claim 4, which is a crystal of (2R,4R)-monatin)₂ magnesium salt having characteristic X-ray diffraction peaks at diffraction angles (2θ±0.2°, CuKα) of 4.9°, 16.8°, 18.0°, and 24.6°.

6. A crystal of a multivalent metal salt of (2R,4R)-monatin according to claim 4, which is selected from the group consisting of:
(1) a crystal of a ((2R,4R)-monatin)₂ magnesium salt having characteristic X-ray diffraction peaks at diffraction angles (2θ±0.2°, CuKα) of 8.7°, 10.5°, 15.9°, 17.4°, 21.0°, and 25.6°,
(2) a crystal of a ((2R,4R)-monatin)₂ magnesium salt having characteristic X-ray diffraction peaks at diffraction angles (2θ±0.2°, CuKα) of 8.9°, 11.2°, 15.0°, 17.8°, and 22.5°; and
(3) a crystal of a ((2R,4R)-monatin)₂ magnesium salt having characteristic X-ray diffraction peaks at diffraction angles (2θ±0.2°, CuKα) of 4.9°, 16.8°, 18.0°, and 24.6°.

7. A crystal of a multivalent metal salt of (2R,4R)-monatin according to claim 4, which is selected from the group consisting of:
(1) a crystal of a ((2R,4R)-monatin)₂ magnesium salt having characteristic X-ray diffraction peaks at diffraction angles (2θ±0.2°, CuKα) of 7.5°, 10.3°, 11.2°, 16.0°, 18.1°, and 23.0°;
(2) a crystal of a ((2R,4R)-monatin)₂ magnesium salt having characteristic X-ray diffraction peaks at diffraction angles (2θ±0.2°, CuKα) of 8.7°, 10.5°, 15.9°, 17.4°, 21.0°, and 25.6°;
(3) a crystal of a ((2R,4R)-monatin)₂ magnesium salt having characteristic X-ray diffraction peaks at diffraction angles (2θ±0.2°, CuKα) of 8.9°, 11.2°, 15.0°, 17.8°, and 22.5°; and
(4) a crystal of a ((2R,4R)-monatin)₂ magnesium salt having characteristic X-ray diffraction peaks at diffraction angles (2θ±0.2°, CuKα) of 4.9°, 16.8°, 18.0°, and 24.6°.

8. A crystal of a multivalent metal salt of (2R,4R)-monatin according to claim 4, which is selected from the group consisting of:
(1) a crystal of a ((2R,4R)-monatin)₂ calcium salt having characteristic X-ray diffraction peaks at diffraction angles (2θ±0.2°, CuKα) of 5.0°, 12.8°, 15.3°, 18.1°, and 23.7°; and
(2) a crystal of a ((2R,4R)-monatin)₂ calcium salt having characteristic X-ray diffraction peaks at diffraction angles (2θ±0.2°, CuKα) of 6.0°, 9.8°, 16.0°, 21.5° and 22.3°.

9. A crystal of a multivalent metal salt of (2R,4R)-monatin according to claim 4, which is selected from the group consisting of:
(1) a crystal of a ((2R,4R)-monatin)₂ calcium salt having characteristic X-ray diffraction peaks at diffraction angles (2θ±0.2°, CuKα) of 5.1°, 15.9°, 19.7°, and 22.3°;
(2) a crystal of a ((2R,4R)-monatin)₂ calcium salt having characteristic X-ray diffraction peaks at diffraction angles (2θ±0.2°, CuKα) of 5.0°, 12.8°, 15.3°, 18.1°, and 23.7°; and
(3) a crystal of a ((2R,4R)-monatin)₂ calcium salt having characteristic X-ray diffraction peaks at diffraction angles (2θ±0.2°, CuKα) of 6.0°, 9.8°, 16.0°, 21.5° and 22.3°.

10. A crystal of a multivalent metal salt of (2R,4R)-monatin according to claim 1, wherein said crystal has an enantiomeric excess of from 10 to 100% ee.

11. A crystal of a multivalent metal salt of (2R,4R)-monatin according to claim 1, wherein said crystal has a diastereomeric excess of from 10 to 100% de.

12. A crystal of a multivalent metal salt of (2R,4R)-monatin according to claim 1, wherein said crystal has a chemical purity of from 50 to 100% by mass.

13. A crystal of a multivalent metal salt of (2R,4R)-monatin according to claim 1, wherein said crystal has a sweetness intensity 200 times or more higher than an aqueous solution of 5% sucrose.

14. A sweetener composition, comprising a crystal of a multivalent metal salt of (2R,4R)-monatin according to claim 1.

15. A sweetener composition according to claim 14, further comprising a reducing sugar.

16. A sweetener composition according to claim 15, wherein said reducing sugar is one or more members selected from the group consisting of dihydroxyacetone, glyceraldehyde, erythrulose, erythrose, threose, ribulose, xylulose, ribose, arabinose, xylose, lyxose, deoxyribose, psicose, fructose, sorbose, tagatose, allose, altrose, glucose, mannose, gulose, idose, galactose, talose, fucose, fucrose, rhamnose, sedoheptulose, lactose, maltose, turanose, cellobiose, maltotriose, and acarbose.

17. A sweetener composition according to claim 14, which is in the form of a powder.

18. A sweetener composition according to claim 14, further comprising a reducing sugar-producing substance.

19. An oral product, comprising a crystal of a multivalent metal salt of (2R,4R)-monatin according to claim 1.

20. An oral product, comprising a sweetener composition according to claim 14.
